(19) United States
(12) Patent Application Publication
(10) Pub. No.: US 2024/0299299 A1
(43) Pub. Date: Sep. 12, 2024

(54) COMPOSITIONS AND METHODS FOR INCREASING THE EFFICACY OF IMMUNOTHERAPIES AND VACCINES

(71) Applicant: The Regents of the University of Michigan, Ann Arbor, MI (US)

(72) Inventors: James J. MOON, Ann Arbor, MI (US); Kai HAN, Ann Arbor, MI (US); Deepak NAGRATH, Ann Arbor, MI (US); Kim HUTCHINGS, Ann Arbor, MI (US); Martin CLASBY, Ann Arbor, MI (US)

(21) Appl. No.: 18/572,884

(22) PCT Filed: Jun. 22, 2022
(86) PCT No.: PCT/US2022/034522

§ 371 Date: Dec. 21, 2023

Related U.S. Application Data
(60) Provisional application No. 63/213,990, filed on Jun. 23, 2021.

(51) Int. Cl. A61K 9/127; A61K 9/00; A61K 9/51; A61K 31/216; A61K 31/235; A61K 31/417; A61K 39/00; A61K 45/06; A61P 35/00; C07K 16/28

(52) U.S. Cl. CPC A61K 9/127; A61K 9/0053; A61K 9/5123; A61K 31/216; A61K 31/235; A61K 31/417; A61K 39/0011; A61K 45/06; A61P 35/00; C07K 16/2818; A61K 2039/505

(57) ABSTRACT

This invention relates generally to compositions and methods for increasing the efficacy of immunotherapies and vaccines. In particular, the present invention relates to compositions comprising one or more metabolites (derivatives, prodrugs, or pharmaceutical salts thereof), and related methods of increasing the efficacy of immunotherapies and vaccines through administration of such compositions.

### STATEMENT OF RELATED APPLICATIONS

This application claims priority to U.S. Provisional Patent Application No. 63/213,990, filed Jun. 23, 2021, the entire contents of which are incorporated herein by reference for all purposes.

### FIELD OF THE INVENTION

This invention relates generally to compositions and methods for increasing the efficacy of immunotherapies and vaccines. In particular, the present invention relates to compositions comprising one or more metabolites (derivatives, prodrugs, or pharmaceutical salts thereof), and related methods of increasing the efficacy of immunotherapies and vaccines through administration of such compositions.

### BACKGROUND OF THE INVENTION

Cancer immunotherapy is revolutionizing the field of oncology. However, immune checkpoint therapies work only in a subset of patients (typically 10-30%). There is a great need to improve the efficacy of immune checkpoint blockade. In addition, there has been extensive research interest to improve vaccines.

The present invention addresses these needs.

### SUMMARY

Gut microbiota is implicated with cancer patients' response rate to immune checkpoint blockers (ICBs)/immune checkpoint inhibitors (ICIs). However, it remains unclear how these gut microorganisms impact the systemic immune responses against distal tumor (see, Ribas, A., et al. Science 2018, 359, 1350-1355). Initial clinical studies have suggested that microbial metabolites, including tryptophan, short-chain-fatty acids, and bile acids, may act as the potential link among gut microbiota, tumor cells, and systemic immune response (see, Ma, C., et al. Science 2018, 360, eaan5931; Botticelli, A., et al. J. Transl. Med. 2020, 18, 1-10; Botticelli, A., et al. Front. Immunol. 2020, 11, 1243). However, current studies are mainly limited to several types of metabolites (see, Sikalidis, A. K. Pathol. Oncol. Res. 2015, 21, 9-17), and the role of many other microbial metabolites in regulating T cell immunity and metabolism is largely unknown. This is partially due to the insufficient metabolic data in vivo (see, Di Biase, S., et al. J. Exp. Med. 2019, 216, 2869-2882). Meanwhile, most of these studies failed to reveal the causal relationship between microbial metabolites and therapeutic efficacy. In particular, it remains unclear whether these beneficial metabolites (also known as postbiotic) can synergize with ICIs (see, Mager, L. F., et al. Science 2020, 369, 1481-1489). Besides, these metabolites with small molecular weight usually require repeated injection at a high dose, due to the very short half-life time (less than hours) in vivo. Thus, the development of microbial metabolite-based postbiotic formulation with controlled release for improved anti-tumor T cell immunity in ICB therapy is highly desirable. In experiments conducted during the course of developing embodiments for the present invention, we (the inventors) screened several metabolites both in vitro and in vivo and demonstrated that some metabolites, including 3,4-dihydroxybenzoate (DHB) and 3-methylhistidine, can induce central memory T cells differentiation, improve T cells survival, and improve antitumor efficacy when combined with ICB. Additional experiments were conducted that utilized the prodrug strategy and developed microbial metabolites prodrug-based formulation, achieving improved anti-tumor T cell immunity during ICB therapy.

Accordingly, the present invention relates generally to compositions and methods for increasing the efficacy of immunotherapies and vaccines. In particular, the present invention relates to compositions comprising one or more metabolites (derivatives, prodrugs, or pharmaceutical salts thereof), and related methods of increasing the efficacy of immunotherapies and vaccines through administration of such compositions. Such compositions and methods are useful for treating cancer, infectious pathogens, autoimmune diseases, neurological disorders, and/or obesity.

In certain embodiments, the present invention provides compositions comprising one or more metabolites (derivatives, prodrugs, or pharmaceutical salts thereof).

Such compositions are not limited to particular type or kind of metabolite. In some embodiments, the metabolite is a naturally occurring metabolite. In some embodiments, the metabolite is non-naturally occurring. In some embodiments, the metabolite is a derivative of a metabolite. In some embodiments, the metabolite is a prodrug form of a metabolite. In some embodiments, the metabolite is a pharmaceutical salt of the metabolite. In some embodiments, the metabolite is any of the metabolites shown in FIG. 1 (see, Example I). In some embodiments, the metabolite is selected from the group consisting of: arginine; 3-methylhistidine; N-acetylneuraminate; disodium sebacate; 3,4-dihydroxybenzoate; pantothenate; γ-aminobutyric acid (GABA); glycerol; AICAR; hippurate; ferulate; creatine; methionine; 1-methylhistidine; acetoin; lithocholate; noradrenaline; oleate; 6-hydroxydopamine; cyclopentanone; trans-cinnamate; 4-acetamidobutanoate; glyceradehyde; 5-valerolactone; 3-dehydroshikimate; xanthurenate; ketoleucine; N-acetylglycine; pipecolate; and N-acetylneuraminate.

In some embodiments, the metabolite is in a prodrug form. In some embodiments, the prodrug form is selected from:

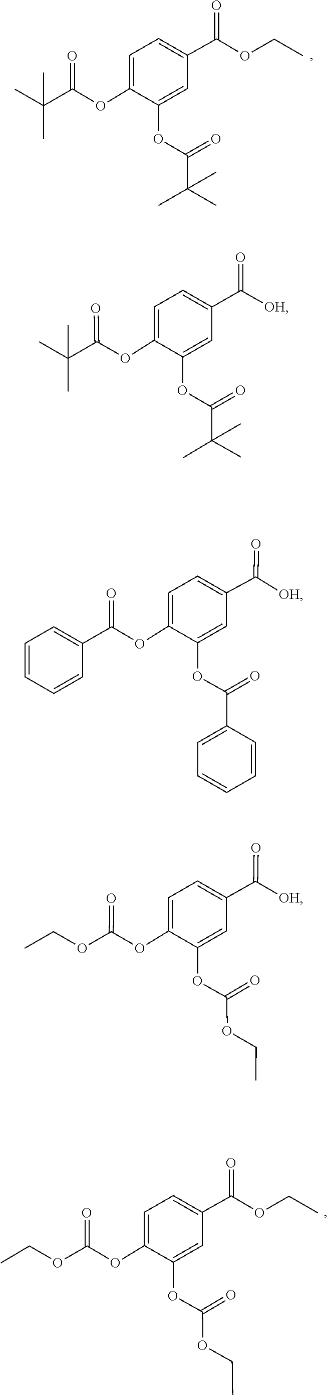

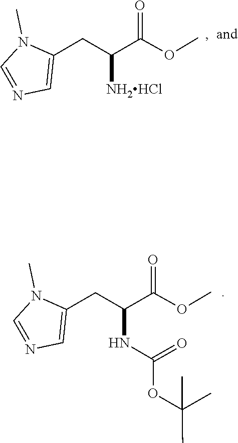

In some embodiments, the metabolite (derivatives, prodrugs, or pharmaceutical salts thereof) comprised within the composition is associated with a biodegradable agent (e.g., a microparticle or a nanoparticle). For example, in some embodiments, the present invention provides compositions comprising biodegradable agents (e.g., microparticles or a nanoparticles) associated with (e.g., complexed, conjugated, encapsulated, absorbed, adsorbed, admixed) such metabolites (derivatives, prodrugs, or pharmaceutical salts thereof).

In some embodiments, biodegradable agent is a microparticle or nanoparticle.

In some embodiments, the size of the microparticle is between 0.5 microns to 100 microns.

In some embodiments, the average size of the nanoparticle is between 6 to 500 nm.

In some embodiments, the nanoparticle is selected from the group consisting of sHDL nanoparticle, fullerenes, endohedral metallofullerenes buckyballs, trimetallic nitride templated endohedral metallofullerenes, single-walled and multi-walled carbon nanotubes, branched and dendritic carbon nanotubes, gold nanorods, silver nanorods, single-walled and multi-walled boron/nitrate nanotubes, carbon nanotube peapods, carbon nanohoms, carbon nanohorn peapods, liposomes, nanoshells, dendrimers, any nanostructures, microstructures, or their derivatives formed using layer-by-layer processes, self-assembly processes, or polyelectrolytes, microparticles, quantum dots, superparamagnetic nanoparticles, nanorods, cellulose nanoparticles, glass and polymer micro- and nano-spheres, biodegradable PLGA micro- and nano-spheres, gold nanoparticles, silver nanoparticles, carbon nanoparticles, iron nanoparticles, a modified micelle, metal-polyhistidine-DOPE@liposome, metal-polyhistidine-PEG, 4arm-PEG-polyhistidine-metal hydrogels, and sHDL-polyhistidine, and metal-organic framework (MOF) coordination polymer (CP).

In some embodiments, the nanoparticle is a sHDL nanoparticle. In some embodiments, the sHDL nanoparticle comprises a mixture of at least one phospholipid and at least one HDL apolipoprotein or apolipoprotein mimetic. In some embodiments, the average particle size of the sHDL nanoparticle is between 6-70 nm.

In some embodiments, the HDL apolipoprotein is selected from the group consisting of apolipoprotein A-I (apo A-I), an ApoA-I mimetic, apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-I milano (apo A-I-milano), apolipoprotein A-I paris (apo A-I-paris), apolipoprotein M (apo M), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

In some embodiments, the phospholipid is selected from the group consisting of dipalmitoylphosphatidylcholine (DPPC), dioleoyl-sn-glycero-3-phosphoethanolamine-N-[3-(2-pyridyldithio) propionate] (DOPE-PDP), 1,2-dipalmitoyl-sn-glycero-3-phosphothioethanol, 1,2-di-(9Z-octadecenoyl)-sn-glycero-3-phosphoethanolamine-N-[4-(p-maleimidophenyl)butyramide], 1,2-dihexadecanoyl-sn-glycero-3-phosphoethanolamine-N-[4-(p-maleimidophenyl)butyramide], 1,2-dihexadecanoyl-sn-glycero-3-phosphoethanolamine-N-[4-(p-maleimidomethyl)cyclohexane-carboxamide], 1,2-di-(9Z-octadecenoyl)-sn-glycero-3-phosphoethanolamine-N-[4-(p-maleimidomethyl)cyclohexane-carboxamide], phosphatidylcholine, phosphatidylinositol, phosphatidylserine, phosphatidylethanolamine, and combinations thereof.

In certain embodiments, the present invention provides compositions comprising a prodrug compound encompassed within Formula I:

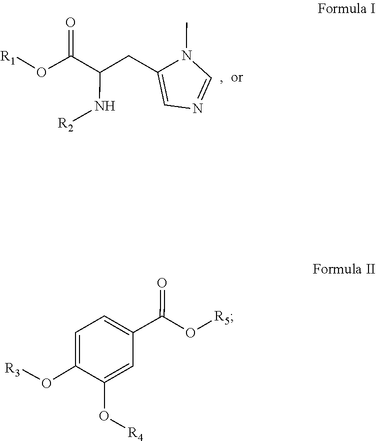

wherein R1 is a chemical moiety selected from alkyl, aryl, heteroaryl, cycloalkyl, cycloheteroaryl, wherein R1 can be substituted with one or more of the following: alkoxy, alkylamino, hydroxy, amino, alkyl, aryl, cycloalkyl, CO₂-alkyl, C(O)NR⁶R⁷,

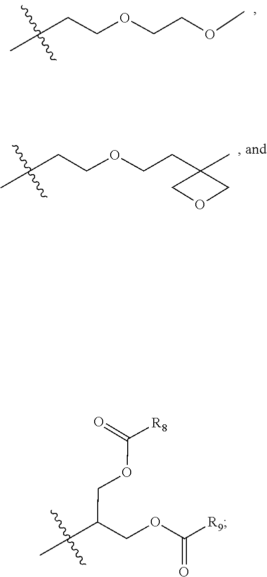

wherein R2 is a chemical moiety selected from hydrogen, C(O)O-alkyl, C(O)O-aryl, C(O)O-cycloalkyl, C(O)alkyl, C(O)aryl, and C(O)cycloalkyl;
wherein R3 is a chemical moiety selected from hydrogen, alkyl, aryl, heteroaryl, cycloalkyl, cycloheteroalkyl, C(O)O-alkyl, C(O)O-aryl, C(O)O-cycloalkyl, C(O)alkyl, C(O)aryl, C(O)cycloalkyl,

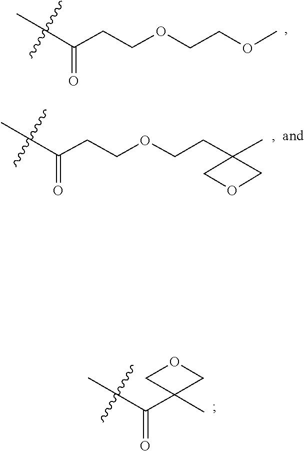

wherein R4 is a chemical moiety selected from hydrogen, alkyl, aryl, heteroaryl, cycloalkyl, cycloheteroalkyl, C(O)O-alkyl, C(O)O-aryl, C(O)O-cycloalkyl, C(O)alkyl, C(O)aryl, C(O)cycloalkyl,

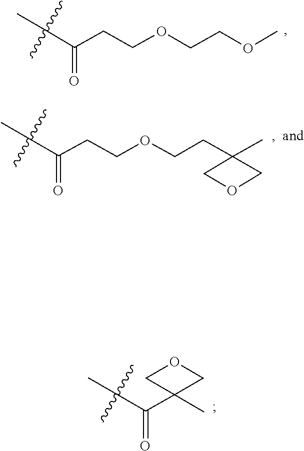

wherein R5 is a chemical moiety selected from alkyl, aryl, heteroaryl, cycloalkyl, cycloheteroaryl, wherein R5 can be substituted with one or more of the following: alkoxy, alkylamino, hydroxy, amino, alkyl, aryl, cycloalkyl, CO₂-alkyl, C(O)NR⁶R⁷,

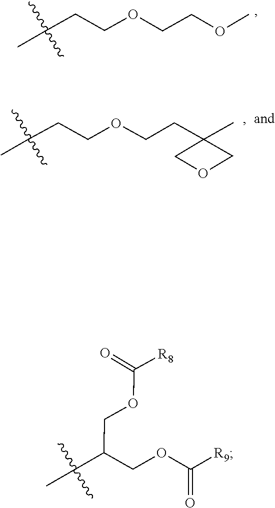

wherein R6 and R7 are each independently selected from the group consisting of hydrogen, alkyl, and cycloalkyl; or wherein R6 and R7 together form a C₄-C₅ alkyl or alkylene chain and together with the nitrogen to which they are attached form a 5 or 6 membered ring;
wherein R8 and R9 are independently selected from the group consisting of alkyl, alkenyl, cycloalkyl, cycloalkenyl, and aryl.

In some embodiments, R1 is methyl.

In some embodiments, R2 is hydrogen or

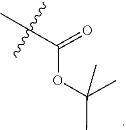 [SMILES: *C(=O)OC(C)(C)C]

In some embodiments, R3 is

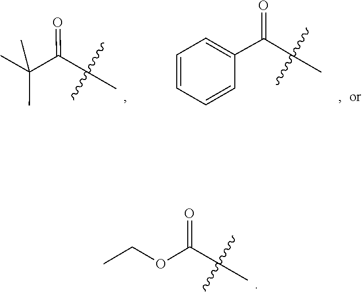

In some embodiments, R4 is

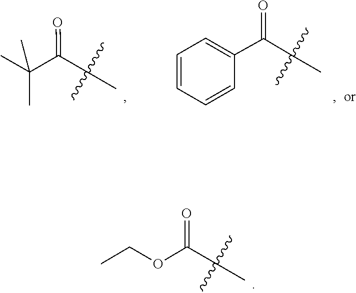

In some embodiments, R5 is hydrogen, or ethyl.

In some embodiments, the prodrug compound is selected from:

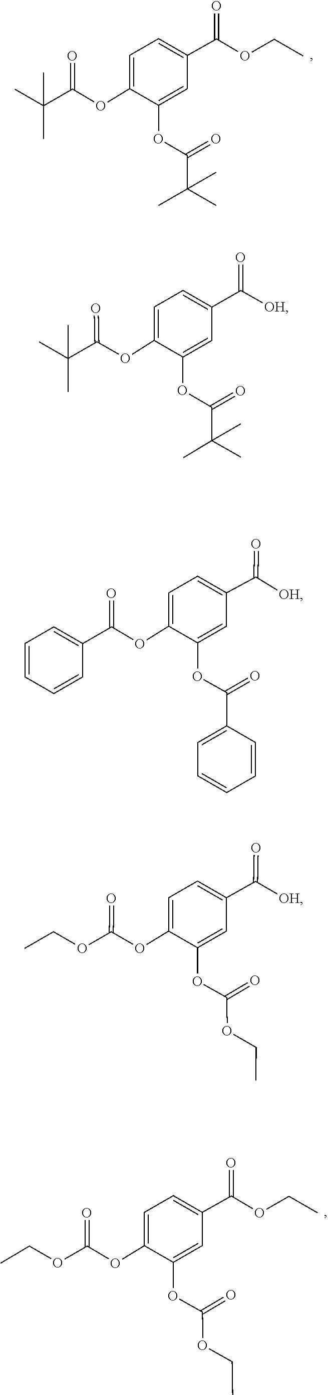

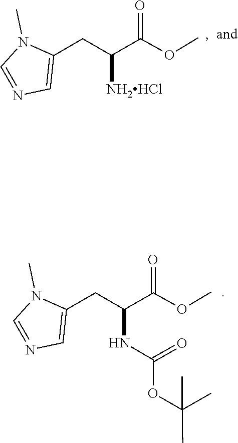

In certain embodiments, the present invention provides a method for increasing the efficacy of a cancer immunotherapy or vaccine (e.g., cancer vaccine) (e.g., vaccines against infectious pathogens) through administration of 1) a cancer immunotherapy or vaccine to a subject, and 2) a composition comprising one or more metabolites (derivatives, prodrugs, or pharmaceutical salts thereof).

In certain embodiments, the present invention provides a method for inhibiting the ability of a cancer cell to induce immune dysfunction, comprising administration of 1) a cancer immunotherapy or cancer vaccine to a subject, and 2) a composition comprising one or more metabolites (derivatives, prodrugs, or pharmaceutical salts thereof).

In certain embodiments, the present invention provides a method for treating or preventing cancer in a subject, comprising administering to the subject 1) a cancer immunotherapy or a cancer vaccine to a subject, and 2) a composition comprising one or more metabolites (derivatives, prodrugs, or pharmaceutical salts thereof).

In certain embodiments, the present invention provides a method for increasing the efficacy of a vaccine through administration of 1) a vaccine to a subject, and 2) administration of a composition comprising one or more metabolites (derivatives, prodrugs, or pharmaceutical salts thereof).

Such methods are not limited to particular manner of administering 1) the cancer immunotherapy or vaccine (e.g., cancer vaccine) (e.g., vaccines against infectious pathogens) to a subject, and 2) a composition comprising one or more metabolites (derivatives, prodrugs, or pharmaceutical salts thereof).

In some embodiments, administration of the composition comprising one or more metabolites (derivatives, prodrugs, or pharmaceutical salts thereof) occurs prior to, concurrent with, and/or after administration of the vaccine (e.g., cancer vaccine) (e.g., vaccines against infectious pathogens) or cancer immunotherapy. In some embodiments, administration of the composition comprising one or more metabolites (derivatives, prodrugs, or pharmaceutical salts thereof) occurs concurrent with administration of the vaccine (e.g., cancer vaccine) (e.g., vaccines against infectious pathogens) or cancer immunotherapy. In some embodiments, administration of the composition comprising one or more metabolites (derivatives, prodrugs, or pharmaceutical salts thereof) occurs prior to administration of the vaccine (e.g., cancer vaccine) (e.g., vaccines against infectious pathogens) or cancer immunotherapy. In some embodiments, administration of the composition comprising one or more metabolites (derivatives, prodrugs, or pharmaceutical salts thereof) occurs prior to and concurrent with administration of the vaccine (e.g., cancer vaccine) (e.g., vaccines against infectious pathogens) or cancer immunotherapy.

Such methods are not limited to a particular type of subject. In some embodiments, the subject is a mammalian subject. In some embodiments, the subject is a human subject.

Such methods are not limited to a particular type of vaccine. In some embodiments, the vaccine is a vaccine for treating cancer, and/or a vaccine for treating and/or protecting from infectious pathogens.

Such methods are not limited to a particular type or kind of cancer immunotherapy. In some embodiments, the cancer immunotherapy comprises one or more immune checkpoint inhibitor (ICI) inhibitors. In some embodiments, the one or more ICI inhibitors are capable of binding to, blocking, and/or inhibit the activity of one or more of CTLA-4, PDL1, PDL2, PD1, BTLA, HVEM, TIM3, GAL9, LAG3, VISTA, KIR, 2B4, CD160 and CGEN-15049. In some embodiments, the one or more ICI inhibitors are selected from Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

Such methods are not limited to particular type or kind of cancer. In some embodiments, the cancer is any type of cancer responsive to cancer immunotherapy or cancer vaccine treatment. In some embodiments, the cancer is one or more of breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

In some embodiments, such methods further comprise administering to the subject one or more chemotherapeutic agents selected from the group consisting of an alkylating agent, an antimetabolite, an anthracycline, an antitumor antibiotic, a monoclonal antibody, a platinum agent, a plant alkaloid, a topoisomerase inhibitor, a vinca alkaloid, a taxane, and an epipodophyllotoxin.

Such methods are not limited to a particular manner of administration of the composition comprising one or more metabolites (derivatives, prodrugs, or pharmaceutical salts thereof). In some embodiments, the composition is administered orally. In some embodiments, the composition is administered by oral gavage. In some embodiments, the composition is administered intratumorally. In some embodiments, the composition is administered intravenously. In some embodiments, the composition is administered subcutaneously.

In some embodiments for such methods, administration of 1) a cancer immunotherapy or vaccine (e.g., cancer vaccine) (e.g., vaccines against infectious pathogens) to a subject, and 2) administration of a composition comprising one or more metabolites (derivatives, prodrugs, or pharmaceutical salts thereof), results in one or more of an increased anti-tumor efficacy of the cancer immunotherapy or vaccine (e.g., cancer vaccine) (e.g., vaccines against infectious pathogens), a stronger immune response (e.g., increased anti-tumor T cell frequency among PBMCs), and enhanced inhibition of tumor growth.

The present invention also provides kits comprising a composition comprising one or more compositions comprising one or more metabolites (derivatives, prodrugs, or pharmaceutical salts thereof), and one or more of a vaccine (e.g., cancer vaccine) (e.g., vaccines against infectious pathogens), and a cancer immunotherapy (e.g., an ICI inhibitor). The kits may optionally contain other therapeutic agents.

### BRIEF DESCRIPTION OF THE DRAWINGS

FIG. 1: CT26 tumor-bearing BALB/c mice were treated with PBS, inulin gel (60 mg per dose), α-PD-1, and inulin gel plus α-PD-1 combo-therapy starting on day 7. α-PD-1 was i.p. injected on days 10, 14 and 18. 255 Metabolites were measured in the feces of the mice on day 21. Shown is the heatmap of the normalized Z-score values of discriminative metabolites. Data represent the mean±s.e.m. (n=5).

FIG. 2: a) OT-I CD8+ T cells were cultured with different metabolites in vitro as indicated, b,c) shown is the frequency of CD44⁺CD62L⁺ Tcm CD8+ T cells on day 8. d) OT-I CD8+ T cells were activated with α-CD3/α-CD28 antibodies in presence of metabolites as indicated and shown is the Tcm CD8+ T cells on day 7. Data represent the mean±s.e.m. (n=5).

FIG. 3: a) OT-I CD8+ T cells were cultured with different metabolites in vitro as indicated, b,c) shown are the frequency of the Tcf1⁺CD8+ T cells and the representative flow cytometry plots on day 6. Data represent the mean±s.e.m. (n=5).

FIG. 4: a) OT-I CD8+ T cells were cultured with different metabolites in vitro as indicated, b,c) shown are the level of survival CD8+ T cells (Annexin V⁻PI⁻) as well as the representative flow cytometry plots on day 5.5. Data represent the mean±s.e.m. (n=5).

FIG. 5: a) Therapeutic treatment regimen. BALB/c mice were s.c. inoculated with 1.5×10⁵ CT26 colon carcinoma cells. The candidate metabolites were injected intratumorally every other day, 100 μg α-PD-1 was i.p. injected on days 9, 12, 16 and 20. b) Average tumor growth, c) individual tumor growth, and d) overall Kaplan-Meier survival are shown. Data represent the mean±s.e.m. (n=5).

FIG. 6: a) Therapeutic treatment regimen. BALB/c mice were s.c. inoculated with 1.5×10⁵ CT26 colon carcinoma cells. The candidate agents were orally administrated 3 times every 4 days, 100 μg α-PD-1 was injected i.p. on days 10, 14, 18 and 22. b) Average tumor growth, c) individual tumor growth. Data represent the mean±s.e.m. (n=4-5).

FIG. 7: a-d) In vitro cell viability of DHB at various concentrations in activated T cells, MC38, B16F10, and CT26 tumor cells. e) BALB/c mice were s.c. inoculated with 1.5×10⁵ CT26 colon carcinoma cells. DHB (2.4 mg per dose) or 3-methylhistidine (3 mg per dose) was i.p. injected 3 times every 4 days, 100 μg α-PD-1 was i.p. injected on days 11, 15, and 20. Shown is the average tumor growth. Data represent the mean±s.e.m. (n=4-5).

FIG. 8: a) The scheme of the synthesis of DHB-based prodrugs. b) The chemical structures of DHB-based prodrugs and 3-methylhistidine-based prodrugs.

FIG. 9: a) Scheme showing the preparation of formulation encapsulating DHB prodrug. b) Therapeutic treatment regimen. BALB/c mice were s.c. inoculated with 1.5×10⁵ CT26 colon carcinoma cells. The prodrug #201 (0.5 mg per dose) or #905 (1 mg per dose) were orally administrated every other day, respectively. 100 μg α-PD-1 was injected i.p. on days 11, 15, and 19. Shown are the (c) average tumor growth; AH1-specific CD8+ T cells in PBMCs on days (d) 18 and (e) 24. Tumor microenvironment analysis: (f) immune cells (CD45⁺ cells) infiltration, (g) CD8⁺ T cells, (h) CD44⁺CD62L⁺ central memory CD8+ T cells, and (i) AH1-specific CD8⁺ T cells in tumor tissue on day 25. Data represent the mean±s.e.m. (n=5).

FIG. 10: BALB/c mice were s.c. inoculated with 1.5×10⁵ CT26 colon carcinoma cells. The prodrug #717 (2 mg per dose) were orally administrated every other day starting day 8. 100 μg α-PD-1 was injected i.p. on days 11 and 16. Shown is the average tumor growth. Data represent the mean±s.e.m. (n=5).

### DEFINITIONS

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising/* or “having” certain elements are also contemplated as “consisting essentially of and “consisting of those certain elements.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The transitional term “comprising,” which is synonymous with “including,” “containing,” or “characterized by,” is inclusive or open-ended and does not exclude additional, unrecited elements or method steps. By contrast, the transitional phrase “consisting of excludes any element, step, or ingredient not specified in the claim. The transitional phrase “consisting essentially of limits the scope of a claim to the specified materials or steps “and those that do not materially affect the basic and novel characteristic(s)” of the claimed invention.

As used herein, the terms “immunce checkpoint blockers” (ICBs), “immune checkpoint inhibitors” (ICIs), “checkpoint inhibitors,” and the like refer to compounds that inhibit the activity of control mechanisms of the immune system. Immune system checkpoints, or immune checkpoints, are inhibitory pathways in the immune system that generally act to maintain self-tolerance or modulate the duration and amplitude of physiological immune responses to minimize collateral tissue damage. ICIs can inhibit an immune system checkpoint by inhibiting the activity of a protein in the pathway. ICI proteins include, but are not limited to, CD80, CD28, CD86, cytotoxic T-lymphocyte-associated protein 4 (CTLA-4), PD-L1, PD-L2, PD-1, Ligand of Inducible T-cell costimulator (L-ICOS), Inducible T-cell co-stimulator (ICOS), CD276, and V-set domain containing T cell activation inhibitor 1 (VTCN1). As such, ICI inhibitors include antagonists of, for example, ICIs such as CTLA4, PD1, or PD-L1. For example, antibodies that bind to CTLA4, PD-1, or PD-L1 and antagonize their function are ICI inhibitors. Moreover, any molecule (e.g., peptide, nucleic acid, small molecule, etc.) that inhibits the inhibitory function of an ICI is an ICI inhibitor.

A “subject” can be a vertebrate, a mammal, or a human. Mammals include, but are not limited to, farm animals, sport animals, pets, primates, mice and rats. In one aspect, a subject is a human.

As used herein, the term “HDL” or “high density lipoprotein” refers to high-density lipoprotein. HDL comprises a complex of lipids and proteins in approximately equal amounts that functions as a transporter of cholesterol in the blood. HDL is mainly synthesized in and secreted from the liver and epithelial cells of the small intestine. Immediately after secretion, HDL is in a form of a discoidal particle containing apolipoprotein A-I (also called apoA-I) and phospholipid as its major constituents, and also called nascent HDL. This nascent HDL receives, in blood, free cholesterol from cell membranes of peripheral cells or produced in the hydrolysis course of other lipoproteins, and forms mature spherical HDL while holding, at its hydrophobic center, cholesterol ester converted from said cholesterol by the action of LCAT (lecithin cholesterol acyltransferase). HDL plays an extremely important role in a lipid metabolism process called “reverse cholesterol transport”, which takes, in blood, cholesterol out of peripheral tissues and transports it to the liver. High levels of HDL are associated with a decreased risk of atherosclerosis and coronary heart disease (CHD) as the reverse cholesterol transport is considered one of the major mechanisms for HDL's prophylactic action on atherosclerosis.

As used herein, the terms “synthetic HDL,” “sHDL,” “reconstituted HDL”, or “rHDL” refer to a particle structurally analogous to native HDL, composed of a lipid or lipids in association with at least one of the proteins of HDL, preferably Apo A-I or a mimetic thereof.

Typically, the components of sHDL may be derived from blood, or produced by recombinant technology.

As used herein, the term “complexed” as used herein relates to the non-covalent interaction of a metabolite (as described herein) with a biodegradable agent (e.g., nanoparticle and/or microparticle).

As used herein, the term “conjugated” as used herein indicates a covalent bond association between a metabolite (as described herein) with a biodegradable agent (e.g., nanoparticle and/or microparticle).

As used herein, the term “encapsulated” refers to the location of a metabolite (as described herein) that is enclosed or completely contained within the inside of a biodegradable agent (e.g., nanoparticle and/or microparticle).

As used herein, the term “absorbed” refers to metabolite (as described herein) that is taken into and stably retained in the interior, that is, internal to the outer surface, of a biodegradable agent (e.g., nanoparticle and/or microparticle).

As used herein, the term “adsorbed” refers to the attachment of a metabolite (as described herein) to the external surface of a biodegradable agent (e.g., nanoparticle and/or microparticle). Such adsorption preferably occurs by electrostatic attraction. Electrostatic attraction is the attraction or bonding generated between two or more oppositely charged or ionic chemical groups. Generally, the adsorption is typically reversible.

As used herein, the term “admixed” refers to a metabolite (as described herein) that is dissolved, dispersed, or suspended in a biodegradable agent (e.g., nanoparticle and/or microparticle). In some cases, the metabolite may be uniformly admixed in the biodegradable agent (e.g., nanoparticle and/or microparticle).

Unless otherwise defined, all technical terms used herein have the same meaning as commonly understood by one of ordinary skill in the art to which this disclosure belongs.

### DETAILED DESCRIPTION

In experiments conducted during the course of developing embodiments for the present invention, we (the inventors) screened several metabolites both in vitro and in vivo and demonstrated that some metabolites, including 3,4-dihydroxybenzoate (DHB) and 3-methylhistidine, can induce central memory T cells differentiation, improve T cells survival, and improve antitumor efficacy when combined with ICB. Additional experiments were conducted that utilized the prodrug strategy and developed microbial metabolites prodrug-based formulation, achieving improved anti-tumor T cell immunity during ICB therapy.

Accordingly, the present invention relates generally to compositions and methods for increasing the efficacy of immunotherapies and vaccines. In particular, the present invention relates to compositions comprising one or more metabolites (derivatives, prodrugs, or pharmaceutical salts thereof), and related methods of increasing the efficacy of immunotherapies and vaccines through administration of such compositions. Such compositions and methods are useful for treating cancer, infectious pathogens, autoimmune diseases, neurological disorders, and/or obesity.

In certain embodiments, the present invention provides compositions comprising one or more metabolites (derivatives, prodrugs, or pharmaceutical salts thereof).

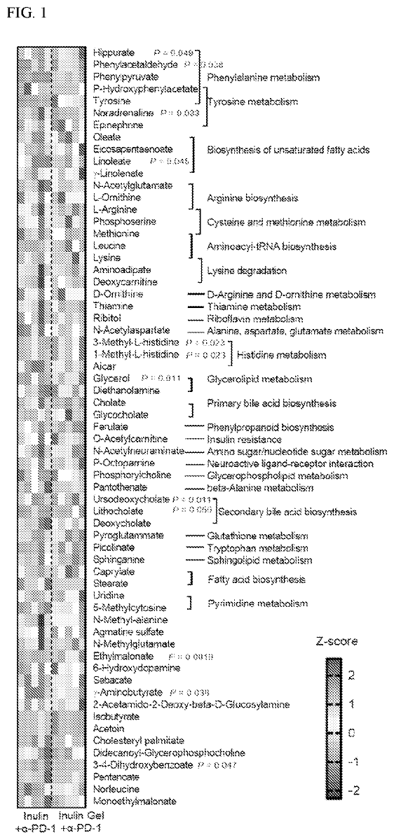

Such compositions are not limited to particular type or kind of metabolite. In some embodiments, the metabolite is a naturally occurring metabolite. In some embodiments, the metabolite is non-naturally occurring. In some embodiments, the metabolite is a derivative of a metabolite. In some embodiments, the metabolite is a prodrug form of a metabolite. In some embodiments, the metabolite is a pharmaceutical salt of the metabolite. In some embodiments, the metabolite is any of the metabolites shown in FIG. 1 (see, Example I). In some embodiments, the metabolite is selected from the group consisting of: arginine; 3-methylhistidine; N-acetylneuraminate; disodium sebacate; 3,4-dihydroxybenzoate; pantothenate; γ-aminobutyric acid (GABA); glycerol; AICAR; hippurate; ferulate; creatine; methionine; 1-methylhistidine; acetoin; lithocholate; noradrenaline; oleate; 6-hydroxydopamine; cyclopentanone; trans-cinnamate; 4-acetamidobutanoate; glyceradehyde; 5-valerolactone; 3-dehydroshikimate; xanthurenate; ketoleucine; N-acetylglycine; pipecolate; and N-acetylneuraminate.

In some embodiments, the metabolite (derivatives, prodrugs, or pharmaceutical salts thereof) comprised within the composition is associated with a biodegradable agent (e.g., a microparticle or a nanoparticle). For example, in some embodiments, the present invention provides compositions comprising biodegradable agents (e.g., microparticles or a nanoparticles) associated with (e.g., complexed, conjugated, encapsulated, absorbed, adsorbed, admixed) such metabolites (derivatives, prodrugs, or pharmaceutical salts thereof).

In some embodiments, the metabolite is in a prodrug form. In some embodiments, the prodrug form is selected from:

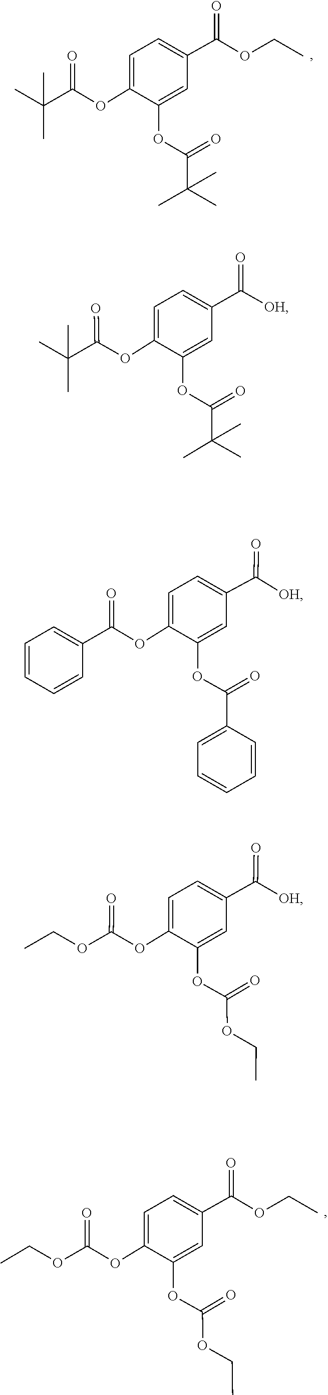

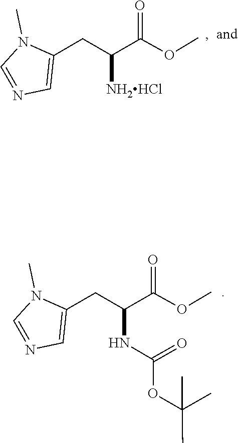

In some embodiments, biodegradable agent is a microparticle or nanoparticle.

In some embodiments, the size of the microparticle is between 0.5 microns to 100 microns.

In some embodiments, the average size of the nanoparticle is between 6 to 500 nm.

In some embodiments, the nanoparticle is selected from the group consisting of sHDL nanoparticle, fullerenes, endohedral metallofullerenes buckyballs, trimetallic nitride templated endohedral metallofullerenes, single-walled and multi-walled carbon nanotubes, branched and dendritic carbon nanotubes, gold nanorods, silver nanorods, single-walled and multi-walled boron/nitrate nanotubes, carbon nanotube peapods, carbon nanohoms, carbon nanohorn peapods, liposomes, nanoshells, dendrimers, any nanostructures, microstructures, or their derivatives formed using layer-by-layer processes, self-assembly processes, or polyelectrolytes, microparticles, quantum dots, superparamagnetic nanoparticles, nanorods, cellulose nanoparticles, glass and polymer micro- and nano-spheres, biodegradable PLGA micro- and nano-spheres, gold nanoparticles, silver nanoparticles, carbon nanoparticles, iron nanoparticles, a modified micelle, metal-polyhistidine-DOPE@liposome, metal-polyhistidine-PEG, 4arm-PEG-polyhistidine-metal hydrogels, and sHDL-polyhistidine, and metal-organic framework (MOF) coordination polymer (CP).

In some embodiments, the nanoparticle is a sHDL nanoparticle. In some embodiments, the sHDL nanoparticle comprises a mixture of at least one phospholipid and at least one HDL apolipoprotein or apolipoprotein mimetic. In some embodiments, the average particle size of the sHDL nanoparticle is between 6-70 nm.

In some embodiments, the HDL apolipoprotein is selected from the group consisting of apolipoprotein A-I (apo A-I), an ApoA-I mimetic, apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-I milano (apo A-I-milano), apolipoprotein A-I paris (apo A-I-paris), apolipoprotein M (apo M), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

In some embodiments, the phospholipid is selected from the group consisting of dipalmitoylphosphatidylcholine (DPPC), dioleoyl-sn-glycero-3-phosphoethanolamine-N-[3-(2-pyridyldithio) propionate] (DOPE-PDP), 1,2-dipalmitoyl-sn-glycero-3-phosphothioethanol, 1,2-di-(9Z-octadecenoyl)-sn-glycero-3-phosphoethanolamine-N-[4-(p-maleimidophenyl)butyramide], 1,2-dihexadecanoyl-sn-glycero-3-phosphoethanolamine-N-[4-(p-maleimidophenyl)butyramide], 1,2-dihexadecanoyl-sn-glycero-3-phosphoethanolamine-N-[4-(p-maleimidomethyl)cyclohexane-carboxamide], 1,2-di-(9Z-octadecenoyl)-sn-glycero-3-phosphoethanolamine-N-[4-(p-maleimidomethyl)cyclohexane-carboxamide], phosphatidylcholine, phosphatidylinositol, phosphatidylserine, phosphatidylethanolamine, and combinations thereof.

In certain embodiments, the present invention provides compositions comprising a prodrug compound encompassed within

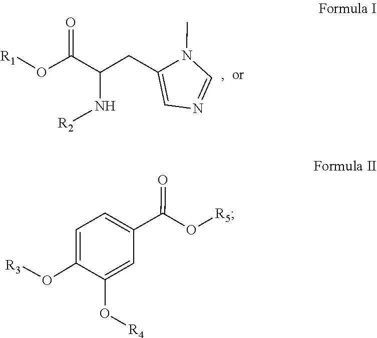

wherein R1 is a chemical moiety selected from alkyl, aryl, heteroaryl, cycloalkyl, cycloheteroaryl, wherein R1 can be substituted with one or more of the following: alkoxy, alkylamino, hydroxy, amino, alkyl, aryl, cycloalkyl, CO₂-alkyl, C(O)NR⁶R⁷,

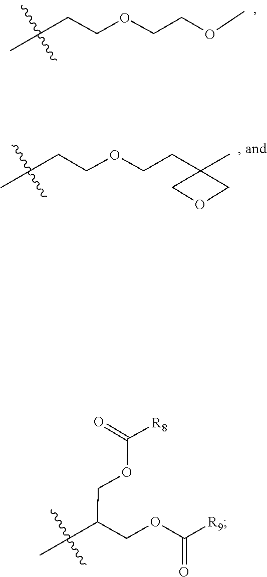

wherein R2 is a chemical moiety selected from hydrogen, C(O)O-alkyl, C(O)O-aryl, C(O)O-cycloalkyl, C(O)alkyl, C(O)aryl, and C(O)cycloalkyl;
wherein R3 is a chemical moiety selected from hydrogen, alkyl, aryl, heteroaryl, cycloalkyl, cycloheteroalkyl, C(O)O-alkyl, C(O)O-aryl, C(O)O-cycloalkyl, C(O)alkyl, C(O)aryl, C(O)cycloalkyl,

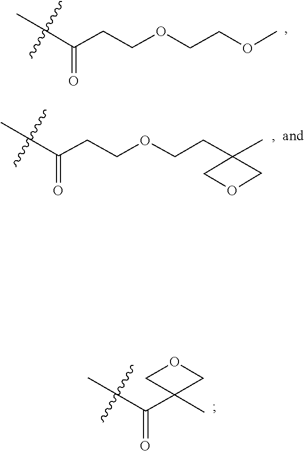

wherein R4 is a chemical moiety selected from hydrogen, alkyl, aryl, heteroaryl, cycloalkyl, cycloheteroalkyl, C(O)O-alkyl, C(O)O-aryl, C(O)O-cycloalkyl, C(O)alkyl, C(O)aryl, C(O)cycloalkyl,

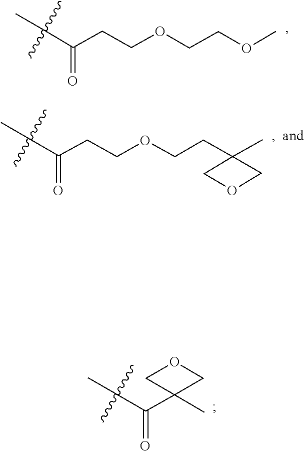

wherein R5 is a chemical moiety selected from alkyl, aryl, heteroaryl, cycloalkyl, cycloheteroaryl, wherein R5 can be substituted with one or more of the following: alkoxy, alkylamino, hydroxy, amino, alkyl, aryl, cycloalkyl, CO₂-alkyl, C(O)NR⁶R⁷,

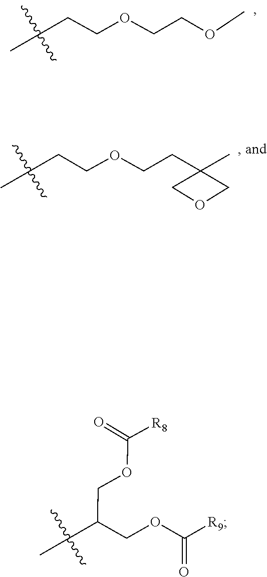

wherein R6 and R7 are each independently selected from the group consisting of hydrogen, alkyl, and cycloalkyl; or wherein R6 and R7 together form a C₄-C₅ alkyl or alkylene chain and together with the nitrogen to which they are attached form a 5 or 6 membered ring;
wherein R8 and R9 are independently selected from the group consisting of alkyl, alkenyl, cycloalkyl, cycloalkenyl, and aryl.

In some embodiments, R1 is methyl.

In some embodiments, R2 is hydrogen or

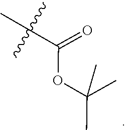 [SMILES: *C(=O)OC(C)(C)C]

In some embodiments, R3 is

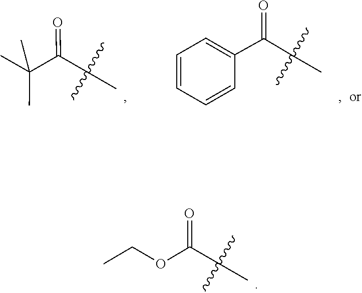

In some embodiments, R3 is

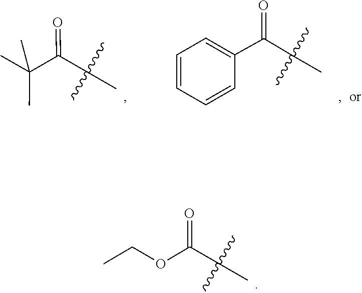

In some embodiments, R5 is hydrogen, or ethyl.

In some embodiments, the prodrug compound is selected from:

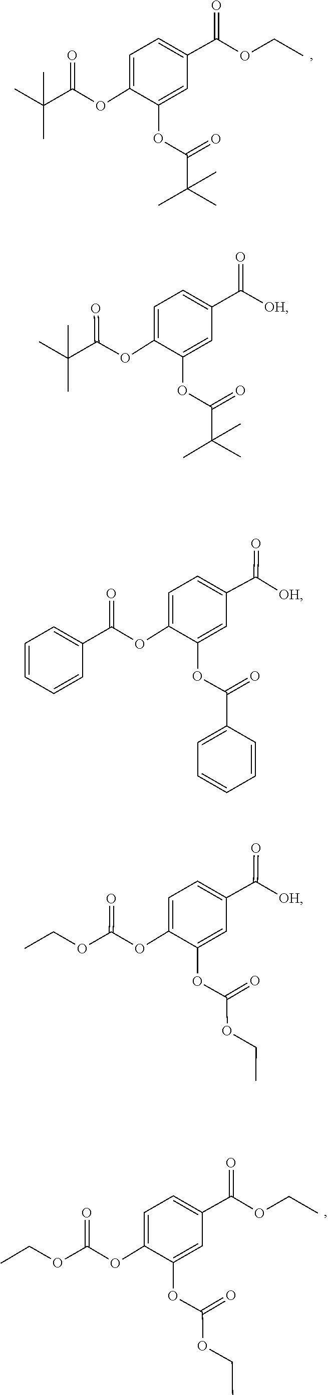

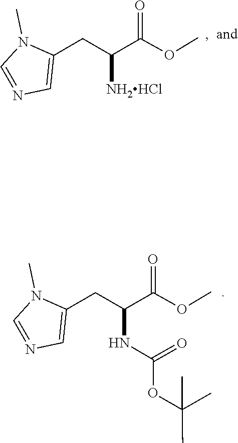

In certain embodiments, the present invention provides a method for increasing the efficacy of a cancer immunotherapy or vaccine (e.g., cancer vaccine) (e.g., vaccines against infectious pathogens) through administration of 1) a cancer immunotherapy or vaccine to a subject, and 2) a composition comprising one or more metabolites (derivatives, prodrugs, or pharmaceutical salts thereof).

In certain embodiments, the present invention provides a method for inhibiting the ability of a cancer cell to induce immune dysfunction, comprising administration of 1) a cancer immunotherapy or cancer vaccine to a subject, and 2) a composition comprising one or more metabolites (derivatives, prodrugs, or pharmaceutical salts thereof).

In certain embodiments, the present invention provides a method for treating or preventing cancer in a subject, comprising administering to the subject 1) a cancer immunotherapy or a cancer vaccine to a subject, and 2) a composition comprising one or more metabolites (derivatives, prodrugs, or pharmaceutical salts thereof).

In certain embodiments, the present invention provides a method for increasing the efficacy of a vaccine through administration of 1) a vaccine to a subject, and 2) administration of a composition comprising one or more metabolites (derivatives, prodrugs, or pharmaceutical salts thereof).

Such methods are not limited to particular manner of administering 1) the cancer immunotherapy or vaccine (e.g., cancer vaccine) (e.g., vaccines against infectious pathogens) to a subject, and 2) a composition comprising one or more metabolites (derivatives, prodrugs, or pharmaceutical salts thereof).

In some embodiments, administration of the composition comprising one or more metabolites (derivatives, prodrugs, or pharmaceutical salts thereof) occurs prior to, concurrent with, and/or after administration of the vaccine (e.g., cancer vaccine) (e.g., vaccines against infectious pathogens) or cancer immunotherapy. In some embodiments, administration of the composition comprising one or more metabolites (derivatives, prodrugs, or pharmaceutical salts thereof) occurs concurrent with administration of the vaccine (e.g., cancer vaccine) (e.g., vaccines against infectious pathogens) or cancer immunotherapy. In some embodiments, administration of the composition comprising one or more metabolites (derivatives, prodrugs, or pharmaceutical salts thereof) occurs prior to administration of the vaccine (e.g., cancer vaccine) (e.g., vaccines against infectious pathogens) or cancer immunotherapy. In some embodiments, administration of the composition comprising one or more metabolites (derivatives, prodrugs, or pharmaceutical salts thereof) occurs prior to and concurrent with administration of the vaccine (e.g., cancer vaccine) (e.g., vaccines against infectious pathogens) or cancer immunotherapy.

Such methods are not limited to a particular subject. In some embodiments, the subject is a mammal. In some embodiments, the subject is a human subject. In some embodiments, the subject is a human subject diagnosed with cancer. In some embodiments, the subject is a human subject at risk for developing cancer.

Such methods are not limited to a specific cancer immunotherapy.

In some embodiments, the cancer immunotherapy is one or more immune checkpoint inhibitors (ICI).

ICIs include any agent that blocks or inhibits in a statistically significant manner, the inhibitory pathways of the immune system. Illustrative ICIs that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. ICIs include antibodies, or antigen binding fragments thereof, other binding proteins, biologic therapeutics or small molecules, that bind to and block or inhibit the activity of one or more of CTLA-4, PDL1, PDL2, PD1, BTLA, HVEM, TIM3, GAL9, LAG3, VISTA, KIR, 2B4, CD160 and CGEN-15049. Illustrative ICIs include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Checkpoint protein ligands include, but are not limited to PD-L1, PD-L2, B7-H3, B7-H4, CD28, CD86 and TIM-3.

In some embodiments, the present invention covers the use of a specific class of ICIs are drugs that block the interaction between immune checkpoint receptor programmed cell death protein 1 (PD-1) and its ligand PD-L1 (see, Mullard, Nature Reviews: Drug Discovery (2013)), 12:489-492. PD-1 is expressed on and regulates the activity of T-cells. Specifically, when PD-1 is unbound to PDL-1, the T-cells can engage and kill target cells. However, when PD-1 is bound to PDL-1 it causes the T-cells to cease engaging and killing target cells. Furthermore, unlike other checkpoints, PD-1 acts proximately such the PDLs are overexpressed directly on cancer cells which leads to increased binding to the PD-1 expressing T-cells.

In some embodiments, ICIs which are antibodies are provided that can act as agonists of PD-1, thereby modulating immune responses regulated by PD-1. In one embodiment, the anti-PD-1 antibodies can be antigen-binding fragments. Anti-PD-1 antibodies disclosed herein are able to bind to human PD-1 and agonize the activity of PD-1, thereby inhibiting the function of immune cells expressing PD-1.

In some embodiments, the present invention covers the use of a specific class of ICIs that are drugs that inhibit CTLA-4. Suitable anti-CTLA4 antagonist agents for use in the methods of the invention, include, without limitation, anti-CTLA4 antibodies, human anti-CTLA4 antibodies, mouse anti-CTLA4 antibodies, mammalian anti-CTLA4 antibodies, humanized anti-CTLA4 antibodies, monoclonal anti-CTLA4 antibodies, polyclonal anti-CTLA4 antibodies, chimeric anti-CTLA4 antibodies, MDX-010 (ipilimumab), tremelimumab, anti-CD28 antibodies, anti-CTLA4 adnectins, anti-CTLA4 domain antibodies, single chain anti-CTLA4 fragments, heavy chain anti-CTLA4 fragments, light chain anti-CTLA4 fragments, inhibitors of CTLA4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281.

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

In one embodiment, the present invention covers the use of a specific class of ICI are drugs that inhibit TIM-3. Blocking the activation of TIM-3 by a ligand, results in an increase in Th1 cell activation. Furthermore, TIM-3 has been identified as an important inhibitory receptor expressed by exhausted CD8+ T cells. TIM-3 has also been reported as a key regulator of nucleic acid mediated antitumor immunity. In one example, TIM-3 has been shown to be upregulated on tumor-associated dendritic cells (TADCs).

Such methods are not limited to the use of a particular cancer vaccine. Indeed, one approach that has been pursued for cancer immunotherapy is the area covered by the term “tumor vaccines” or “cancer vaccines” which includes immunization with tumor specific or overexpressed antigens. In this approach, an antigen or antigens specific for, or overexpressed in, tumor cells are injected alone, with adjuvants, as part of a microorganism that delivers the antigen (for example, Listeria monocytogenes), or after incubation ex-vivo with immune cells (including but not limited to dendritic cells) in order to elicit cellular and/or humoral immune responses.

In some embodiments, the cancer is carcinoma. Carcinomas are cancers of epithelial origin. In some embodiments, the carcinoma is selected from the group consisting of acinar carcinoma, acinous carcinoma, alveolar adenocarcinoma, carcinoma adenomatosum, adenocarcinoma, carcinoma of adrenal cortex, alveolar carcinoma, alveolar cell carcinoma, basal cell carcinoma, carcinoma basocellular, basaloid carcinoma, basosquamous cell carcinoma, breast carcinoma, bronchioalveolar carcinoma, bronchiolar carcinoma, cerebriform carcinoma, cholangiocellular carcinoma, chorionic carcinoma, colloid carcinoma, comedocarcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliate adenoids, carcinoma exulcere, carcinoma fibrosum, gelatinform carcinoma, gelatinous carcinoma, giant cell carcinoma, gigantocellulare, glandular carcinoma, granulose cell carcinoma, hair matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lentivular carcinoma, carcinoma lenticulare, lipomatous carcinoma, lymphoepithelial carcinoma, carcinoma mastotoids, carcinoma medullare, medullary carcinoma, carcinoma melanodes, melanotonic carcinoma, mucinous carcinoma, carcinoma muciparum, carcinoma mucocullare, mucoepidermoid carcinoma, mucous carcinoma, carcinoma myxomatodes, masopharyngeal carcinoma, carcinoma nigrum, oat cell carcinoma, carcinoma ossificans, osteroid carcinoma, ovarian carcinoma, papillary carcinoma, periportal carcinoma, preinvasive carcinoma, prostate carcinoma, renal cell carcinoma of kidney, reserve cell carcinoma, carcinoma sarcomatodes, scheinderian carcinoma, scirrhous carcinoma, carcinoma scrota, signet-ring cell carcinoma, carcinoma simplex, small cell carcinoma, solandoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberrosum, tuberous carcinoma, verrucous carcinoma, carcinoma vilosum.

In some embodiments, the cancer is a sarcoma. Sarcomas are mesenchymal neoplasms that arise in bone and soft tissues. In some embodiments, the sarcoma is selected from liposarcomas (including myxoid liposarcomas and pleomorphic liposarcomas), leiomyosarcomas, rhabdomyosarcomas, neurofibrosarcomas, malignant peripheral nerve sheath tumors, Ewing's tumors (including Ewing's sarcoma of bone, extraskeletal or non-bone) and primitive neuroectodermal tumors (PNET), synovial sarcoma, hemangioendothelioma, fibrosarcoma, desmoids tumors, dermatofibrosarcoma protuberance (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) and osteosarcoma (also known as osteogenic sarcoma-skeletal and extra-skeletal, and chondrosarcoma.

In some embodiments, the cancer is a refractory or a responding cancer. As used herein, a refractory cancer is a cancer that is resistant to the ordinary standards of care prescribed. These cancers, although initially responsive to treatment, recur and/or may be completely non responsive to the treatment.

In some embodiments, the cancer is an immunogenic cancer. Examples of immunogenic cancers include malignant melanoma and renal cell carcinoma, Mantel cell lymphoma, follicular lymphoma, diffuse large B-cell lymphoma, T-cell acute lymphoblastic leukemia, Burkitt Lymphoma, myeloma, immunocytoma, acute promyelocyte leukemia, chronic myeloid/acute lymphoblastic leukemia, acute leukemia, B-cell acute lymphoblastic leukemia, anaplastic large cell leukemia, myelodysplasia syndrome/acute myeloid leukemia, non-Hodgkin's lymphoma, chronic lymphocytic leukemia, acute myelogenous leukemia (AML), common (pre-B) acute lymphocytic leukemia, malignant melanoma, T-cell lymphoma, leukemia, B-cell lymphoma, epithelial malignancies, lymphoid malignancies, gynecologic carcinoma, biliary adenocarcinomas and ductal adenocarcinomas of the pancreas.

In some embodiments, such methods further comprise administering other therapies such as, for example, radiation therapy, surgery, conventional chemotherapy or with a combination of one or more additional therapies. Such other active ingredient includes, but is not limited to glutathione antagonists, angiogenesis inhibitors, chemotherapeutic agent(s) and antibodies (e.g., cancer antibodies). The agents described in this invention may be administered simultaneously or sequentially. The separation in time between administrations may be minutes, hours, days or it may be longer.

Such methods are not limited to use of a particular chemotherapeutic and/or cytotoxic agents. Examples include, but are not limited to, alkylating agents (e.g., chlorambucil, cyclophosphamide, ccnu, melphalan, procarbazine, thiotepa, bcnu, and busulfan), antimetabolites (e.g., 6-mercaptopurine and 5-fluorouracil), anthracyclines (e.g., daunorubicin, doxorubicin, idarubicin, epirubicin, and mitoxantrone), antitumor antibiotics (e.g., bleomycin), monoclonal antibodies (e.g., alemtuzumab, bevacizumab, cetuximab, gemtuzumab, ibritumomab, panitumumab, rituximab, tositumomab, and trastuzumab), platinums (e.g., cisplatin, oxaliplatin, and carboplatin), plant alkaloids (e.g., vincristine), topoisomerase I or II inhibitors (e.g., irinotecan, topotecan, amsacrine, etoposide, etoposide phosphate, and teniposide), vinca alkaloids (e.g., vincristine, vinblastine, vinorelbine, and vindesine), taxanes (e.g., paclitaxel and docetaxel), epipodophyllotoxins (e.g., etoposide and teniposide), nucleoside analogs, and angiogenesis inhibitors (e.g., Avastin (beracizumab), a humanized monoclonal antibody specific for VEGF-A).

Examples of glutathione antagonists include but are not limited to buthionine sulfoximine, cyclophosphamide, ifosphamide, actinomycin-d and N-(4-hydroxyphenyl) retinamide (4-HPR). Examples of angiogenesis inhibitors include but are not limited to 2-methoxyestradiol(2-ME), AG3340, Angiostatin, antithrombin-III, Anti-VEGF antibody, Batimastat, bevacizumab (Avastin), BMS-275291, CA1, Canstatin, combretastatin, Combretastatin-A4 phosphate, CC-5013, captopril, celecoxib, Dalteparin, EMD121974, Endostatin, Erlotinib, Gefitinib, Genistein, Halofuginone, ID 1, ID3, IM862, Imatinib mesylate, Inducible protein-10, Interferon-alpha, Interleukin-12, Lavendustin-a, LY317615, or AE-941, Marimastat, Mapsin, Medroxyprogesterone acetate, Meth-1, Meth-2, Neovastat, Osteopontin cleaved product, PEX, Pigment epithelium growth factor (PEGF), platelet growth factor 4, prolactin fragment, proliferin-related protein (PRP), PTK787/ZK222584, recombinant human platelet factor-4(rPF4), restin, squalamine, SU5416, SU6668, Suramin, Taxol, Tecogalan, Thalidomide, Tetrathiomolybdate (TM), Thrombospondin, TNP-470, Troponin I, Vasostatin, VEGF1, VEGF-TPvAP and ZD6474. In some embodiment the angiogenesis inhibitor is a VRGF antagonist. The VEGF antagonist may be a VEGF binding molecule. VEGF binding molecule include VEGF antibodies, or antigen binding fragment (s) thereof. One example of a VEGF antagonist is NeXstar.

Examples of categories of chemotherapeutic agents that may be used in any of the methods or agents disclosed herein include, but are not limited to, DNA damaging agents and these include topoisomerase inhibitors (e.g., etoposide, camptothecin, topotecan, irinotecan, teniposide, mitoxantrone), anti-microtubule agents (e.g., vincristine, vinblastine), antimetabolite agents (e.g., cytarabine, methotrexate, hydroxyurea, 5-fluorouracil, flouridine, 6-thioguanine, 6-mercaptompurine, fludarabine, pentostatin, chlorodeoxyadenosine), DNA alkylating agents (e.g., cisplatin, mecholorethamine, cyclophosphamide, ifosphamide, melphalan, chlorambucil, busulfan, thiotepa, carmustine, lomustine, carboplatin, dacarbazine, procarbazine) and DNA strand break inducing agents (e.g., bleomycin, doxorubicin, daunorubicin, idarubicin, mitomycin C).

Chemotherapeutic agents include synthetic, semisynthetic and naturally derived agents. Important chemotherapeutic agents include, but are not limited to, Avicine, Aclarubicin, Acodazole, Acronine, Adozelesin, Adriamycin, aldesleukin, Alitretinoin, AUopurinol sodium, Altretamine, Ambomycin, Ametantrone acetate, Aminoglutethimide, Amsacrine, Anastrazole, Annonaceous Acetogenins, Anthramycin, Asimicin, Asparaginase, asperlin, Azacitidine, azetepa, Azotomycin, batimastat, benzodepa, bexarotene, Bicalutamide, Bisantrene, Bisnafide, Bizelesin, Bleomycin, Brequinar, Bropirimine, Bullatacin, Busulfan, Cabergoline, cactinomycin, calusterone, caracemide, carbetimer, carboplatin, carmustine, carubicin, carzelesin, cedefingol, chlorambucil, celecoxib, cirolemycin, cisplatin, cladribine, crisnatol, cyclophosphamide, cytarabine, dacarbazine, DACA, dactinomycin, Daunorubicin, daunomycin, Decitabine, denileukin, Dexormaplatin, Dezaguanine, Diaziquone, Docetaxel, Doxorubicin, Droloxifene, Dromostalone, Duazomycin, Edatrexate, Eflornithine, Elsamitrucin, Estramustine, Etanidazole, Etoposide, Etoprine, Fadrozole, Fazarabine, Fenretinide, Floxuridine, Fludarabine, Fluorouracil, Flurocitabine, 5-FdUMP, Fosquidone, Fosteuecine, FK-317, FK-973, FR-66979, FR-900482, Gemcitabine, Gemtuzumab, Ozogamicin, Gold Aul 98, Goserelin, Guanacone, Hydroxyurea, Idarubicin, Ilmofosine, Interferon alpha and analogs, Iproplatin, irinotecan, Lanreotide, Letrozole, Leuprolide, Liarozole, Lometrexol, Lomustine, Losoxantrone, masoprocol, Maytansine, Mechlorethamine, Megestrol, Melengestrol, Melphalan, Menogaril, Metoprine, maturedepa, mitindomide, Mitocarcin, Mitogillin, Mitomalacin, Mitomycin, Mitomycin C, Mitosper, Mitotane, Mitoxantrone, Mycophenolic acid, Nocodazole, Nogalamycin, Oprelvekin, ormaplatin, Oxisuran, Paclitaxel, pamidronate, pegaspargase, Peliomycin, Pentamustine, Peplomycin, Perfosfamide, Pipobroman, Piposulfan, Piroxantrone, Plicamycin, Plomestane, Porfimer, Porfiromycin, Prednimustine, procarbazine, Puromycin, Pyrazofurin, Riboprine, Rituximab, Rogletimide, Rolliniastatin, safingol, Samarium, Semustine, Simtrazene, Sparfosate, Sparsomycin, spirogermanium, Spiromustine, Spiroplatin, Squamocin, Squamotacin, streptonigrin, streptozocin, SrC12, Sulphofenur, Talisomycin, Taxane, Toxoid, Tecoglan, Tegafur, teloxantrone, Temoporfin, teniposide, Teroxirone, Testolactone, Thiamiprine, Thiotepa, Thymitaq, Tiazofurin, Tirapazamine, Tomudex, Top-53, Topotecan, Toremixifme, Trastuzumab, Trestolone, triciribine, Triciribine, Trimetrexate, trimetrexate glucuronate, Triptorelin, Tubulozole, uracil mustard, Uredepa, valrubicin, vapreotide, Vinblastine, Vincristine, Vindesine, Vinepidine, Vinglycinate, Vinleurosine, Vinorelbine, Vinrosidine, Vinzolidine, Vorozole, Zeniplatin, Zinostatin, Zorubicin, 2-cholrodeoxyrubicine, 2′-deoxyformycin, 9-aminocamptothecin, raltitrexed, N-propargyl-5,8-didezafolic acid, 2-cholo-2′arabinofluoro-2′ deoxyadenosine, 2-cholo-2′-deoxyadenosine, anisomycin, Trichostatin, hPRL-G129R, CEP-751, Linomide, Sulfur mustard, nitrogen mustard, N-methyl-N-nitrosourea, fotemustine, Streptozotocin, dacarbazine, mitozolomide, temozolomide, AZQ, ormaplatin, CI-973, DWA21 14R, JM216, JM335, Bisplatinum, Tomudex, azacitidine, cytrabincine, gemcitabine, 6-mercaptopurine, Hypoxanthine, Teniposide, CPT-11, Doxorubicin, Daunorubicin, Epirubicin, darubicin, losoxantrone, amsacrine, pyrazoloacridine, all trans retinol, 14-hydroxy-retro-retinol, all-trans retinoic acid, N-(4-hydroxyphenyl) retinamide, 13-cisretinoic acid, 3-methyl TTNEB, 9-cisretenoic acid, fludarabine, and 2-Cda.

Other chemotherapeutic agent include: 20-epi1,25-dihydroxyvitamin-D3, 5-ethynyl uracil, abiraterone, aclarubicin, acylfulvene, adecylpenol, adozelesin, aldesleukin, ALL-TK antagonists, altretamine, ambumastine, amidox, amifostine, amino levulinic acid, anagrelide, anastrozole, andrographolide, angiogenesis inhibitors, antagonist D, antagonists D, antarelix, anti-dorsalizing morphogenetic protein-1, antiandrogen, antiestrogen, antineoplastone, antisense oligonucleotides, aphidicolin, apoptosis gene modulators, apoptosis regulators, apurinic acid, ara-cdp-dl-PTBA, arginine aminase, asulacrine, atamestine, atrimustine, axinamastine 1 and axinamastine 2, axinamastine 3, azasetron, azatoxin, azatyrosine, baccatin III derivatives, balanol, BCR/ABL antagonist, benzochlorins, benzoylsaurosporine, beta lactam derivatives, beta-alethine, perillyl alcohol, phenozenomyein, phenyl acetate, phosphatase inhibitors, picibanil, pilocarbine and salts or analogs thereof, pirarubucin, piritrexim, placetin A, placetin B, plasminogen activator inhibitor, platinum complex, phenyl ethyl isothiocyanate and analogs thereof, platinum compounds, platinum triamine complex, podophylotoxin, porfimer sodium, porphyromycin, propyl bis acridones, prostaglnadins J2, protease inhibitors, protein A based immune modulators, PKC inhibitors, microalgal, protein tyrosine phosphatase inhibitors, purine nucleoside phosphorylase inhibitors, purpurins, pyrazoloacridines, pyridoxylated hemoglobin polyoxyethylene conjugate, raf antagonists, raltitrexed, ramosetron, ras famesyl protein tranferase inhibitors, rasinhibitors, ras-GAP inhibitors, ratellitptine demethylated, Rhenium Re 186 etidronate, rhizoxine, ribozyme, RII retinide, rogletimide, rosagliatazone and analogs and derivatives thereof, rohitukine, romurtide, roquinimex, rubiginone B1, ruboxyl, safingol, saintopin, SarCNU, sarcophytol A, sargrmostim, sdi 1 mimetics, semustine, senescence derived inhibitor 1, sense oligonucleotide, signal transduction inhibitors, signal transduction modulators, single chain antigen binding protein, sizofiran, sobuzoxane, sodium borocaptate, sodium phenyl acetate, solverol, somatomedin binding protein, sonermin, sparfosic acid, spicamycin D, spiromustin, splenopentine, spongistatin 1, squalamine, stem cell inhibitor, stem cell division inhibitor, stipiamide, stromelysin, sulfinosine, superactive vasoactive intestinal peptide antagonists, suradista, siramin, swainsonine, synthetic glycosaminogly cans, tallimustine, tamoxifen methiodide, tauromustine, tazarotene, tacogalan sodium, tegafur, tellurapyrilium, telomerase inhibitors, temoporfin, tmeozolomide, teniposide, tetrachlorodecaoxide, tetrazomine, thaliblastine, thalidomide, thiocoraline, thrombopoetin and mimetics thereof, thymalfasin, thymopoetin receptor agonist, thymotrinan, thyroid stimulating harmone, tin ethyl etiopurpin, tirapazamine, titanocene and salts thereof, topotecan, topsentin, toremifene, totipotent stem cell factors, translation inhibitors, tretinoin, triacetyluridine, tricribine, trimetrexate, triptorelin, tropisetron, turosteride, tyrosine kinase inhibitors, tyrphostins, UBC inhibitors, ubenimex, urogenital sinus derived growth inhibitory factor, urokinase receptor antagonists, vapreotide, variolin B, vector system, erythrocyte gene therapy, velaresol, veramine, verdins, verteporfin, vinorelbine, vinxaltine, vitaxin, vorozol, zanoterone, zeniplatin, zilascorb and zinostatin.

Other chemotherapeutic agents include: antiproliferative agents (e.g., piritrexim isothiocyanate), antiprostatic hypertrophy agents (sitogluside), Benign prostatic hyperplasia therapy agents (e.g., tomsulosine, RBX2258), prostate growth inhibitory agents (pentomone) and radioactive agents: Fibrinogen 1125, fludeoxyglucose F18, Flurodopa F18, Insulin 1125, Iobenguane 1123, Iodipamide sodium 1131, Iodoantipyrine 1131, Iodocholesterol 1131, Iodopyracet 1125, Iofetamine HCL 1123, Iomethin 1131, Iomethin 1131, Iothalamate sodium 1125, Iothalamate 1131, Iotyrosine 1131, Liothyronine 1125, Merosproprol Hgl 97, Methyl ioodobenzo guanine (MIBG-1131 or MIBGI 123) selenomethionine Se75, Technetium Tc99m furifosmin, technetium Tc99m gluceptate, Tc99m Biscisate, Tc99m disofenin, TC99m gluceptate, Tc99m lidofenin, Tc99m mebrofenin, Tc99m medronate and sodium salts thereof, Tc99m mertiatide, Tc99m oxidronate, Tc99m pentetate and salts thereof, Tc99m sestambi, Tc99m siboroxime, Tc99m succimer, Tc99m sulfur colloid, Tc 99m teboroxime, Tc 99m Tetrofosmin, Tc99m Tiatide, Thyroxine 1125, Thyroxine 1131, Tolpovidone 1131, Triolein 1125 and Treoline 1125, and Treoline 131, MIBG-1123 and MIBG 1131 are especially preferred chemotherapeutic agents for co-administration with the nitrofuran containing pharmaceutical composition of invention.

Another category of chemotherapeutic agents are anticancer supplementary potentiating agents, e.g., antidepressant drugs (Imipramine, desipramine, amitriptyline, clomipramine, trimipramine, doxepin, nortriptyline, protriptyline, amoxapine, and maprotiline), or no-trycyclic anti-depressant drugs (sertraline, trazodone and citalopram), Ca++ antagonists (verapamil, nifedipine, nitrendipine and caroverine), calmodulin inhibitors (prenylamine, trifluoperazine and clomipramine), Amphotericin B, Triparanol analogs (e.g., Tamoxifen), antiarrhythmic drugs (e.g., quinidine), antihypertensive drugs (e.g., reserpine), thiol depleters (e.g., buthionine and sulfoximine) and multiple drug resistance reducing agents such as Cremophor EL.

In some embodiments, the condition characterized with dysregulated gut microbiome activity is an autoimmune disease, a neurological disorder, diabetes, and/or obesity. Examples of such conditions include, but are not limited to, rheumatoid arthritis, multiple sclerosis diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

Such methods are not limited to a particular manner of administration of the composition comprising one or more metabolites (derivatives, prodrugs, or pharmaceutical salts thereof). In some embodiments, the composition is administered orally. In some embodiments, the composition is administered by oral gavage. In some embodiments, the composition is administered intratumorally. In some embodiments, the composition is administered intravenously. In some embodiments, the composition is administered subcutaneously. However, administration can be by any suitable route of administration including buccal, dental, endocervical, intramuscular, inhalation, intracranial, intralymphatic, intramuscular, intraocular, intraperitoneal, intrapleural, intrathecal, intratracheal, intrauterine, intravascular, intravenous, intravesical, intranasal, ophthalmic, otic, biliary perfusion, cardiac perfusion, priodontal, rectal, spinal subcutaneous, sublingual, topical, intravaginal, transermal, ureteral, or urethral. Dosage forms can be aerosol including metered aerosol, chewable bar, capsule, capsule containing coated pellets, capsule containing delayed release pellets, capsule containing extended release pellets, concentrate, cream, augmented cream, suppository cream, disc, dressing, elixer, emulsion, enema, extended release fiber, extended release film, gas, gel, metered gel, granule, delayed release granule, effervescent granule, chewing gum, implant, inhalant, injectable, injectable lipid complex, injectable liposomes, insert, extended release insert, intrauterine device, jelly, liquid, extended release liquid, lotion, augmented lotion, shampoo lotion, oil, ointment, augmented ointment, paste, pastille, pellet, powder, extended release powder, metered powder, ring, shampoo, soap solution, solution for slush, solution/drops, concentrate solution, gel forming solution/drops, sponge, spray, metered spray, suppository, suspension, suspension/drops, extended release suspension, swab, syrup, tablet, chewable tablet, tablet containing coated particles, delayed release tablet, dispersible tablet, effervescent tablet, extended release tablet, orally disintegrating tablet, tampon, tape or troche/lozenge.

Intraocular administration can include administration by injection including intravitreal injection, by eyedrops and by trans-scleral delivery.

Administration can also be by inclusion in the diet of the mammal such as in a functional food for humans or companion animals.

The specific dose can be calculated according to the approximate body weight or body surface area of the patient or the volume of body space to be occupied. The dose will also depend upon the particular route of administration selected. Further refinement of the calculations necessary to determine the appropriate dosage for treatment is routinely made by those of ordinary skill in the art. Such calculations can be made without undue experimentation by one skilled in the art in light of the activity in assay preparations such as has been described elsewhere for certain compounds (see for example, Howitz et al., Nature 425:191-196, 2003 and supplementary information that accompanies the paper). Exact dosages can be determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

The present invention also provides kits comprising an agent capable of elevating the richness and diversity of the subject's gut microbiome (e.g., a fiber based pre-biotic), and one or more of a vaccine (e.g., a cancer vaccine) (e.g., a vaccine for treating and/or protecting from infectious pathogens), and a cancer immunotherapy (e.g., an ICI inhibitor). The kits may optionally contain other therapeutic agents.

### EXPERIMENTAL

The following examples are provided to demonstrate and further illustrate certain preferred embodiments of the present invention and are not to be construed as limiting the scope thereof.

### Example I

In previous studies, we (the inventors) demonstrated that inulin gel administered orally synergized with α-PD-1 immune checkpoint blocker (ICB) slowed down tumor growth. We therefore conducted new experiments that examined changes in the metabolites in tumor-bearing mice undergoing inulin gel plus α-PD-1 IgG combo-therapy. We examined 255 metabolites in feces and found that inulin gel plus α-PD-1 IgG combo-therapy substantially altered the levels of many microbial metabolites (FIG. 1). Some of these metabolites have been already reported to improve the efficacy of immune checkpoint blockers (ICBs), and they include creatine and methionine (see, Di Biase, S., et al. J. Exp. Med. 2019, 216, 2869-2882; Bian, Y., et al. Nature 2020, 585, 277-282). We also observed significant changes in the levels of hippurate, 1-methylhistidine, 3-methylhistidine, and oleate. Notably, prior clinical studies have reported that patients who respond to α-PD-1 IgG therapy have higher content of hippurate, 1-methylhistidine, 3-methylhistidine, and oleate (see, Hatae, R., et al. JCI insight 2020, 5, e133501); however, it remains to be seen whether these metabolites can actually improve the efficacy of ICBs.

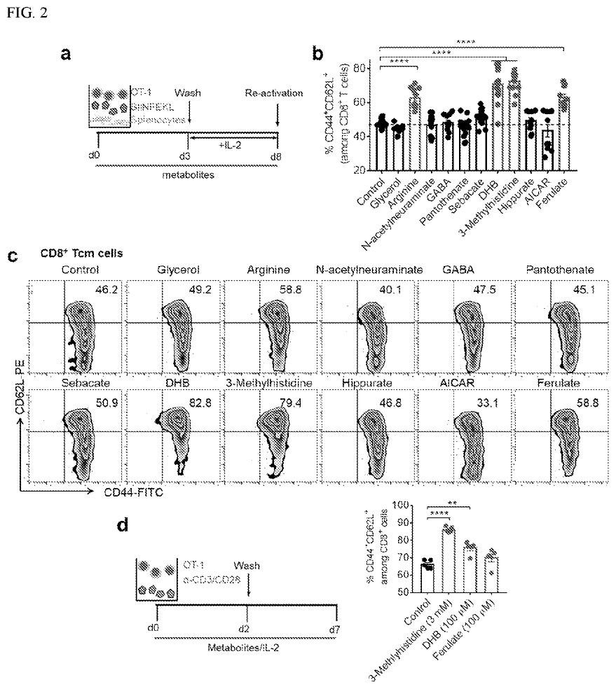

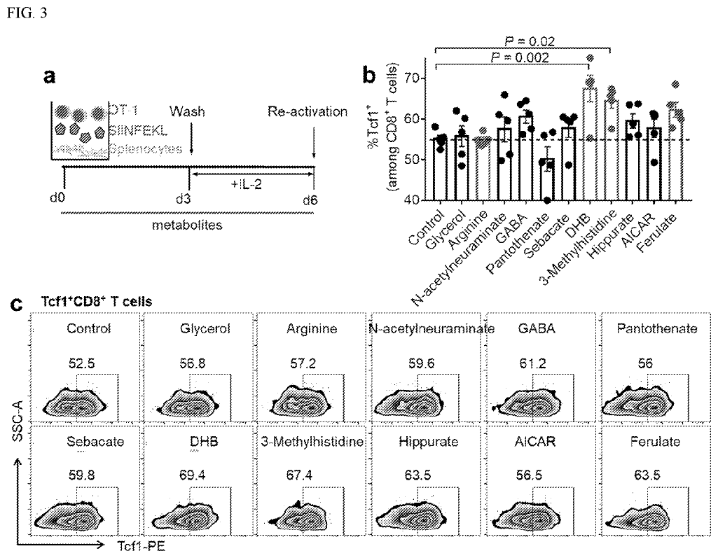

In addition, it remains unclear how these microbial metabolites affect T cell fate in terms of differentiation, survival, and polyfunctional cytokine expression. Central memory CD8⁺ T cells (Tcm) represent a subset of T cells that could effectively control the tumor growth. We examined whether microbial metabolites could induce Tcm differentiation during activation of CD8⁺ T cells. From metabolites that underwent significant changes upon inulin gel therapy, we chose candidate metabolites with good water solubility. OT-I CD8⁺ T cells from spleen were incubated with candidate metabolites and activated with antigen peptide in the presence of interleukin-2 (IL-2) (FIG. 2). Interestingly, L-arginine, 3-methylhistidine, sodium ferulate and 3,4-dihydroxybenzoate (DHB)-supplemented medium significantly increased the differentiation of CD44⁺CD62L⁺ central memory CD8⁺ T cells on day 8 (FIG. 2a-c). Meanwhile, 3-methylhistidine and DHB can also induce central memory CD8⁺ T cells in the absence of dendritic cells (FIG. 2d). Moreover, 3-methylhistidine, sodium ferulate and DHB increased the frequency of Tcf1⁺CD8⁺ T cells on day 6 (FIG. 3), these T cells are associated with the positive outcome of ICB in cancer patients (see, Jansen, C. S., et al. Nature 2019, 576, 465).

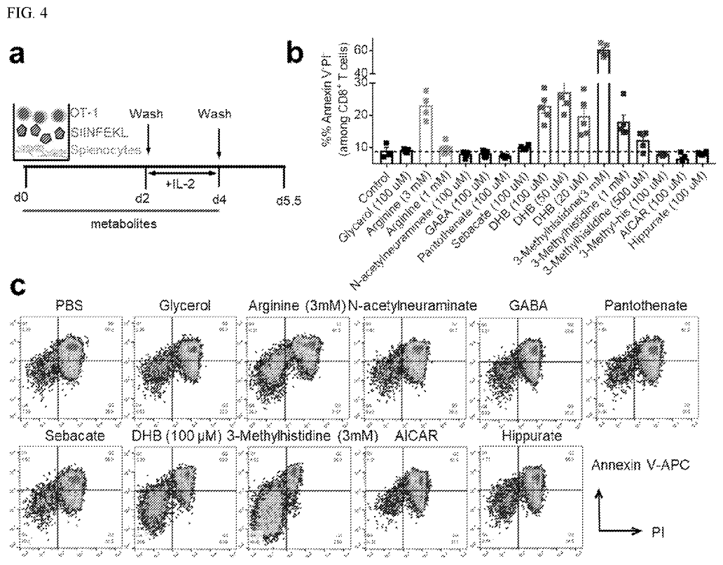

We also assessed whether microbial metabolites could affect CD8⁺ T cell survival. After expanding CD8⁺ T cells in the presence of candidate metabolites and IL-2, the viability of CD8⁺ T cells was measured upon IL-2 withdrawal. L-Arginine, 3-methylhistidine and DHB supplementation significantly increased the survival of activated CD8⁺ T cells, and this improved survival of CD8⁺ T cells was also dependent on the dose of metabolites (FIG. 4).

We next assessed if 3-methylhistidine and DHB could improve the antitumor efficacy of ICB in vivo. Here, L-arginine is not included as L-arginine has already been reported to improve the antitumor effect in vivo (see, Geiger, R., et al. Cell 2016, 167, 829-842). In addition, we assessed hippurate, sebacate and ferulate. Prior studies showed that clinical responders to ICB therapy have higher concentration of hippurate in comparison with non-responders (see, Hatae, R., et al. JCI insight 2020, 5, e133501). In contrast, the concentration of hippurate decreased dramatically in some cancer patients in comparison to healthy humans (see, Tan, G., et al. Sci. Rep. 2017, 7, 46176). Ferulic acid, a phenolic compound, can induce apoptosis in cancer cells (see, Eroglu, C., et al. Tumor Biol. 2015, 36, 9437-9446), while sebacate as a dicarboxylate can undergo β-oxidation, tend to accumulate and be excreted when normal fatty acid metabolism is disrupted (see, Liu, G., et al. J. Biol. Chem. 1996, 271, 25338-25344). Overall, it remains unknown whether these metabolites can synergize with ICBs.

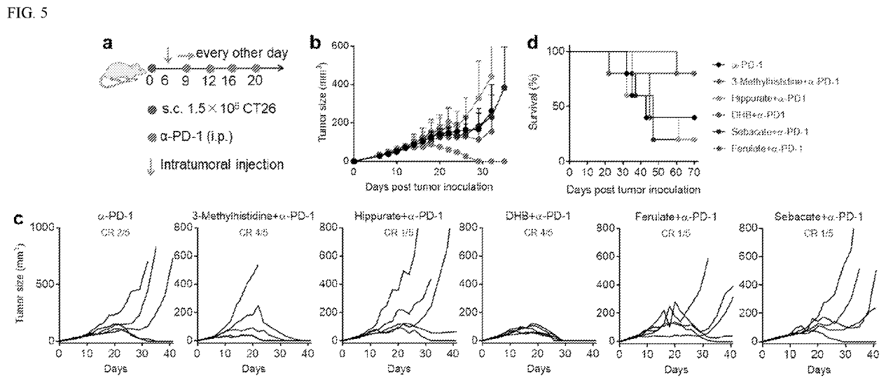

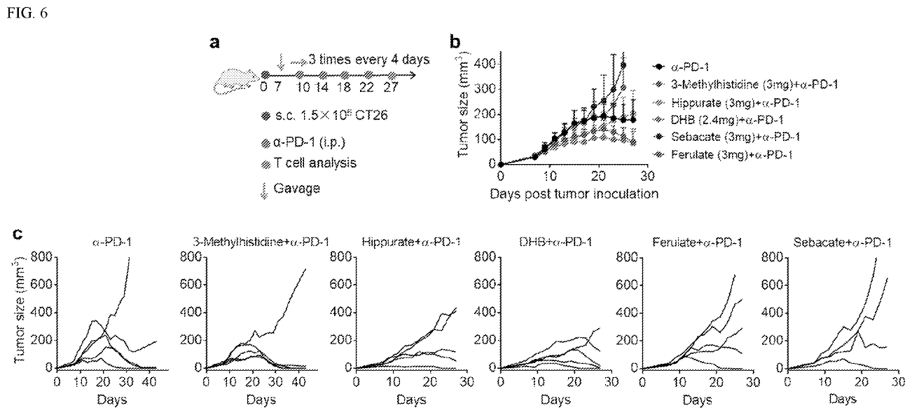

BALB/c mice were subcutaneously (s.c.) inoculated in their flank with CT26 colon carcinoma cells on day 0. Systemic intraperitoneal (i.p.) administration of α-PD-1 IgG was performed at preset days (FIG. 5a, FIG. 6a). The metabolites were intratumorally or orally injected. In intratumoral injection treatment, DHB and 3-methylhistidine improved the systemic antitumor efficacy of α-PD-1 treatment and slowed down the CT26 tumors growth (FIG. 5b-c). 80% of the mice in DHB and 3-methylhistidine groups remained tumor-free on day 70, compared with 40% tumor-free mice in α-PD-1 monotherapy group (FIG. 5d).

We also tested these metabolites administered via oral gavage. When combined with α-PD-1 IgG administered i.p., orally administered DHB and 3-methylhistidine improved the efficacy of α-PD-1 treatment (FIG. 6).

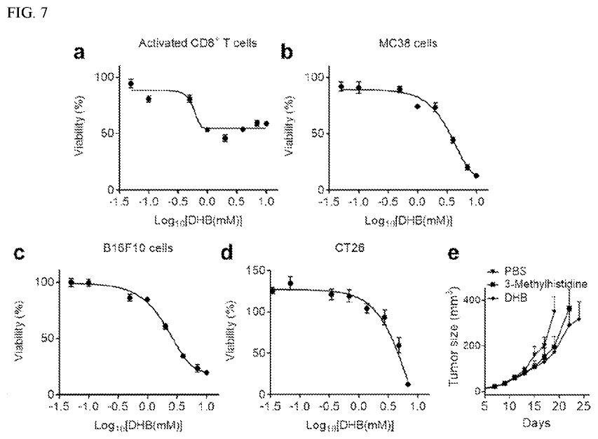

In addition, DHB exhibited low cell toxicity when the concentration was below 1 mM. DHB or 3-methylhistidine monotherapy without α-PD-1 IgG did not inhibit the tumor growth in vivo (FIG. 7), suggesting that DHB and 3-methylhistidine do not directly kill cancer cells.

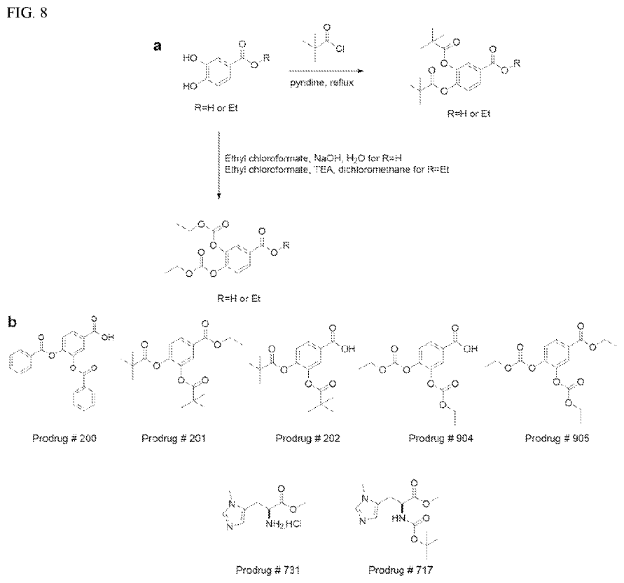

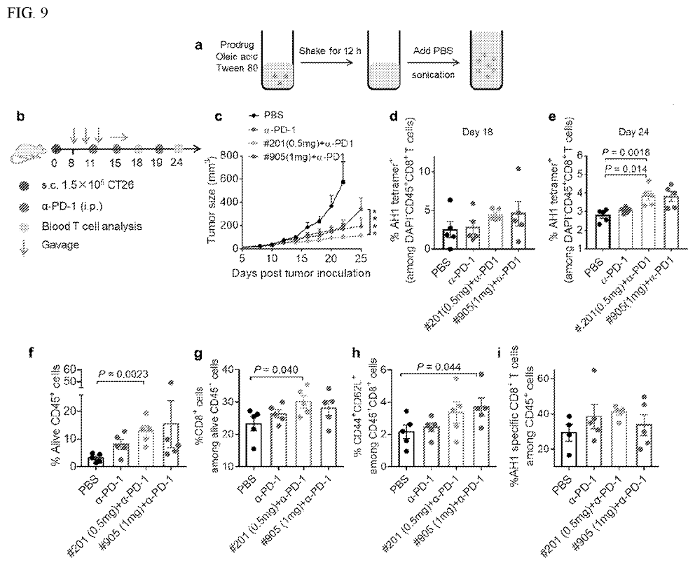

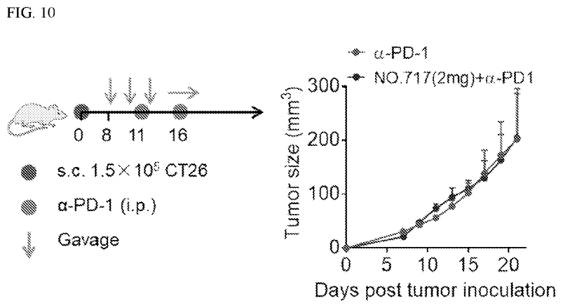

Due to the small molecular weight and high hydrophilicity, the metabolites that we tested are thought to be cleared rapidly in vivo (see, Chen, W., et al. J. Chromatogr. B 2012, 908, 39-44; Long, C. L., et al. Metabolism 1975, 24, 929-935). On the other hand, their prodrugs are anticipated to improve their pharmacokinetics. According to the in vitro and in vivo screening studies, we focused on the development of DHB or 3-methylhistidine based prodrugs. Thus, we designed five DHB-based prodrugs and two 3-methylhistidine-based prodrugs (FIG. 8). We formulated DHB-based prodrugs in lipid-based dose forms (FIG. 9a). We found that only prodrug #201 and #905 can be successfully encapsulated. Further in vivo analysis showed that orally administered formulation containing prodrug #201 plus α-PD-1 substantially slowed down the tumor growth and increased the frequency of AH1-specific CD8⁺ T cells among the peripheral blood mononuclear cells (PBMCs) (FIG. 9b-e). The formulation containing prodrug #201 plus α-PD-1 also promoted the infiltration of immune cells (CD45⁺ cells), especially the AH1-specific CD8⁺ T cells and CD44⁺CD62L⁺ central memory CD8⁺ T cells in tumor tissue (FIG. 9f-i). We also tested 3-methylhistidine-based prodrug #717, but we found that prodrug #717 did not synergize with α-PD-1 against tumor growth in vivo (FIG. 10).

### Example II

This example describes the methods described in Example I.

In Vitro Differentiation/Survival of OT-I T Cells with Various Metabolites.

Splenocytes from OT-I mice were obtained aseptically. The cells were cultured in 96-well plates (1×10⁵ cells per well). For Tcm differentiation study, cells were activated with SIINFEKL peptide (4×10⁻⁹M) in the presence of various metabolites (arginine: 3 mM; 3-methylhistidine: 3 mM; N-acetylneuraminate: 100 μM; disodium sebacate: 100 μM; DHB: 100 μM; pantothenate: 100 μM; γ-aminobutyric acid: 100 μM; glycerol: 100 μM; AICAR: 100 μM, sodium ferulate: 100 μM) for 3 days. Then cells were washed three times with T-cell medium and further cultured with IL-2 (10 ng ml⁻¹; PeproTech) in the presence of metabolites for 5 days. The cells were then washed twice and re-activated with SIINFEKL peptide (10 μM) for 2 h. The suspension cells were collected, blocked with CD16/32 antibody and stained with the fixable viability dye eFluor 450, FITC-CD44 rat anti-human/mouse and PE-CD62L monoclonal antibody. To test if the central memory CD8+ T cells differentiation is dependent on dendritic cells, CD8 T cells isolated from the spleen in OT-I mice were incubated in the 96-well plate, which was precoated with anti-hamster IgG antibody. β-CD3/β-CD28 (1 μg mL⁻¹) and IL-2 (100 U mL⁻¹) were added into each well in the presence of 3-methylhistidine (3 mM), DHB (100 μM), or sodium ferulate (100 μM) for 2 days. Then cells were washed for three times to remove α-CD3/α-CD28 antibodies and further incubated with metabolites and IL-2 (10 ng ml⁻¹) for 5 days. Thereafter, cells were collected for central memory CD8⁺ T cells analysis. For CD8⁺ T cells survival study, cells were activated with antigen peptide as above, following with thoroughly washing, and incubation in presence of IL-2 for 2 days. Metabolites were added during this incubation (arginine: 3 mM, 1 mM; 3-methylhistidine: 3 mM, 1 mM, 500 μM, 100 μM; N-acetylneuraminate: 100 μM; disodium sebacate: 100 μM; DHB: 100 μM, 50 μM, 20 μM; pantothenate: 100 μM; γ-aminobutyric acid: 100 μM; glycerol: 100 μM; AICAR: 100 μM). Then cells were further incubated in absence of both IL-2 and metabolites for 36 h to induce the apoptosis/dead. For Tcf1⁺CD8⁺ differentiation study, cells were activated with antigen peptide as above, followed by thoroughly washing, and incubation with metabolites in presence of IL-2 for 3 days. Cells were stained with PE-Tcf1/Tcf7 rabbit monoclonal antibody (clone C63D9). Tcf1⁺CD8⁺, CD44⁺CD62L⁺CD8⁺ and survival (stained with Annexin V-APC and PI) T cells were detected via flow cytometry.

### In Vivo Cancer Immunotherapy of Metabolites Plus α-PD-1.

Animals were cared for following the federal, state and local guidelines. All work conducted on animals was in accordance with and approved by the Institutional Animal Care and Use Committee (IACUC). Female (6-8 weeks old) BALB/c mice from Jackson Laboratory were inoculated with 1.5×10⁵ CT26 cells per mouse on the right flank by s.c. injection. Tumor-bearing mice were randomly assigned to different treatment groups. For intratumoral injection study, the mice were injected with 3-methylhistidine (dose: 1 mg per mouse), sodium hippurate (dose: 1 mg per mouse), disodium sebacate (dose: 1 mg per mouse), sodium ferulate (dose: 1 mg per mouse), or DHB (dose: 0.5 mg per mouse) in 40 μL PBS buffer for every other day. The mice were i.p. injected with α-PD-1 antibody (100 μg per dose; clone RMP1-14, Bioxcell) on days 9, 12, 16 and 20 post tumor inoculation. For oral administration study, the mice were injected with 3-methylhistidine (dose: 3 mg per mouse), sodium hippurate (dose: 3 mg per mouse), disodium sebacate (dose: 3 mg per mouse), sodium ferulate (dose: 3 mg per mouse), or DHB (dose: 2.4 mg per mouse) in PBS buffer for 3 times every 4 days. α-PD-1 was injected on days 10, 14, 18 and 22. The tumor size was measured, and the tumor volume was calculated as ½×(length×width²). Tumor-bearing mice were euthanized when the tumor size reached 1.5 cm in any diameter or when the animals became moribund with severe weight loss (>20%) or tumor ulceration.

### Preparation of Lipid Based Formulation Containing DHB Prodrug.

The DHB-based prodrugs were synthesized by the Medicinal Chemistry Core in University of Michigan. To prepare the formulation, the prodrug #201 or #905 (4 mg) was added to the mixture of oleic acid (45 μL) and Tween 80 (25 μL) and incubated overnight to equilibrate and allow the prodrug to dissolve. Then an aqueous phase consisting of PBS buffer was added, and the formulation emulsified by ultrasonication with an ultrasonic processor.

### In Vivo Cancer Immunotherapy of Prodrug Based Formulation Plus α-PD-1.

The CT26 tumor model was established as above. The formulation containing prodrug #201 or #905 was orally administered starting on day 8 and performed every other day. α-PD-1 antibody (100 μg per dose) was i.p. injected on days 11, 15, and 19. The PBMCs were obtained on days 18 and 24 and stained with PE-tagged peptide-MHC tetramer (H-2L^{d}-restricted SPSYVYHQF). Mice were euthanized on day 25 and tumor tissues were cut into small pieces and incubated with collagenase type IV (1 mg ml-1) and DNase I (0.1 mg ml-1) under gentle shaking. After 30 min, the cell suspension was filtered through a 70-μm strainer. The cells were washed with fluorescence-activated cell sorting (FACS) buffer and blocked with CD16/32 antibody. The cells were then stained with the designated antibodies: efluor450, FITC-CD44 rat anti-human/mouse, PE-CD62L monoclonal antibody, and APC-CD8 rat anti-mouse, PE-Tcf1/Tcf7 rabbit monoclonal antibody, APC-efluor780-Cd45 Rat anti Mouse (Clone: 30 F11), PE-AH1-specific tetramer. Cells were analyzed by flow cytometry.

### Example III

This example describes the methods for synthesizing prodrugs that were evaluated in Example I and II.

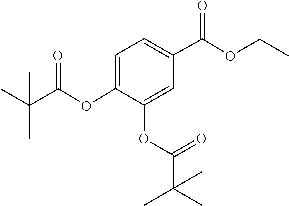 [SMILES: CCOC(=O)c1ccc(OC(=O)C(C)(C)C)c(OC(=O)C(C)(C)C)c1]

Prodrug #201 [4-(ethoxycarbonyl)-1,2-phenylene bis(2,2-dimethylpropanoate)]: To a solution of ethyl 3,4-dihydroxybenzoate (1.5 g, 8.2 mmol) in pyridine (20 mL) was added pivaloyl chloride (2.2 mL, 18 mmol). The reaction was heated at reflux for 2.5 h. The reaction was cooled to room temperature, quenched with 1N HCl, then extracted 3× with ethyl acetate. Combined organic layers were dried over magnesium sulfate, filtered, and concentrated. The crude residue was purified by column chromatography eluting with 0-20% ethyl acetate in hexanes to afford the title compound as a colorless oil (2.6 g, 92%). HRMS (ESI) m/z: Calculated for [M+Na]⁺ 373.1627; Found: 373.1615.

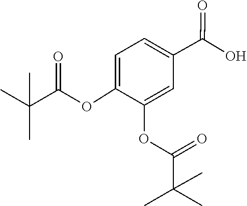 [SMILES: CC(C)(C)C(=O)Oc1ccc(C(=O)O)cc1OC(=O)C(C)(C)C]

Prodrug #202 [3,4-bis(pivaloyloxy)benzoic acid]: To a solution of 3,4-dihydroxybenzoic acid (1.5 g, 9.7 mmol) in pyridine (20 mL) was added pivaloyl chloride (2.6 mL, 21 mmol). The reaction was heated at reflux for 3 h. The reaction was cooled to room temperature, quenched with 1N HCl, then extracted 3× with ethyl acetate. The combined organic layers were dried over magnesium sulfate, filtered, and concentrated. The crude residue was purified by column chromatography eluting with 20-70% ethyl acetate in hexanes. Fractions containing product were combined, dissolved in dichloromethane, and washed with 1N HCl to remove the residual pyridine. The organic layer was dried over magnesium sulfate, filtered, and concentrated to give the title compound as a white solid (420 mg, 13%). HRMS (ESI) m/z: Calculated for [M+Na]+345.1344; Found: 345.1299.

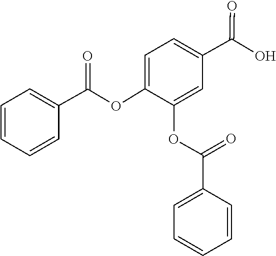 [SMILES: O=C(O)c1ccc(OC(=O)c2ccccc2)c(OC(=O)c2ccccc2)c1]

Prodrug #200 [3,4-bis(benzoyloxy)benzoic acid]: The synthesis followed the previous report (see, Saito, Y., et al. Org. Lett. 2018, 20, 628-631).

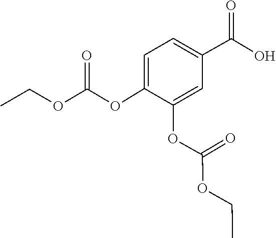 [SMILES: CCOC(=O)Oc1ccc(C(=O)O)cc1OC(=O)OCC]

Prodrug #904 [3,4-bis((ethoxycarbonyl)oxy)benzoic acid]: To a suspension of 3,4-dihydroxybenzoic acid (2.0 g, 13 mmol) in water (40 mL) was added sodium hydroxide (1.7 g, 42 mmol). The reaction mixture was cooled to 0° C. and ethyl chloroformate (2.7 mL, 29 mmol) was slowly added. Stirred at 0° C. for 1.5 h. The reaction mixture was acidified with 50 mL of 1N HCl. The resulting precipitate was filtered, washed with water, and dried overnight on vacuum. The material was then purified by column chromatography eluting with 0-5% methanol in dichloromethane with 0.1% HOAc to give the title compound as a white solid (863 mg, 22%). HRMS (ESI) m/z: Calculated for [M+Na]+321.0586; Found: 321.0569.

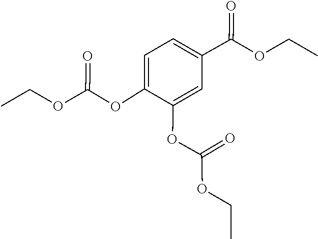 [SMILES: CCOC(=O)Oc1ccc(C(=O)OCC)cc1OC(=O)OCC]

Prodrug #905 [ethyl 3,4-bis((ethoxycarbonyl)oxy)benzoate]: To a suspension of ethyl 3,4-dihydroxybenzoate (0.30 g, 1.6 mmol) in dichloromethane (8 mL) was added triethylamine (1.1 mL, 8.2 mmol) dropwise. The reaction mixture was cooled to 0° C. and ethyl chloroformate (0.79 mL, 8.2 mmol) was added dropwise. After stirring for 4 h, the reaction was diluted with methylene chloride and washed with saturated aqueous sodium bicarbonate solution and brine. The organic layer was dried over magnesium sulfate, filtered, and concentrated. The crude residue was purified by column chromatography eluting with 0-20% ethyl acetate in hexanes. Isolated 574 mg of a clear colorless liquid. The impure oil was dissolved in dichloromethane and washed with 1N HCl, saturated sodium bicarbonate solution and brine. The organic layer was dried over magnesium sulfate, filtered, and concentrated to give the title compound as a colorless oil (499 mg, 92%). HRMS (ESI) m/z: Calculated for [M+Na]+349.0899; Found: 349.0813.

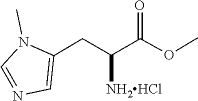

Prodrug #731 [methyl Np-methyl-L-histidinate hydrochloride]: The synthesis followed the previous report (see, Nakamura, O., et al. JP2009046476).

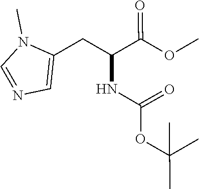 [SMILES: COC(=O)[C@H](Cc1cncn1C)NC(=O)OC(C)(C)C]

Prodrug #717 [methyl Na-(tert-butoxycarbonyl)-Np-methyl-L-histidinate]: The synthesis followed the previous report (see, Nakamura, O., et al. JP2009046476).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A composition comprising one or more metabolites; derivatives, prodrugs, or pharmaceutical salts thereof.

2. The composition of claim 1, wherein the at least one of the one or more metabolites is
a naturally occurring metabolite; and/or
selected from the group consisting of: arginine; 3-methylhistidine; N-acetylneuraminate; disodium sebacate; 3,4-dihydroxybenzoate; pantothenate; γ-aminobutyric acid (GABA); glycerol; AICAR; hippurate; ferulate; creatine; methionine; 1-methylhistidine; acetoin; lithocholate; noradrenaline; oleate; 6-hydroxydopamine; cyclopentanone; trans-cinnamate; 4-acetamidobutanoate; glyceradehyde; 5-valerolactone; 3-dehydroshikimate; xanthurenate; ketoleucine; N-acetylglycine; pipecolate; and N-acetylneuraminate; and/or
is shown in FIG. 1; and/or
is a prodrug compound encompassed within Formula I: 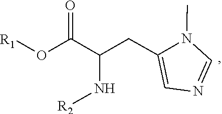
or Formula II: 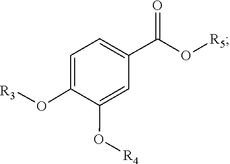
wherein R1 is a chemical moiety selected from alkyl, aryl, heteroaryl, cycloalkyl, cycloheteroaryl, wherein R1 can be substituted with one or more of the following: alkoxy, alkylamino, hydroxy, amino, alkyl, aryl, cycloalkyl, CO₂-alkyl, C(O)NR⁶R⁷, 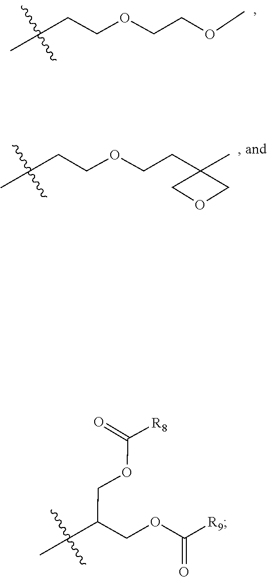
wherein R2 is a chemical moiety selected from hydrogen, C(O)O-alkyl, C(O)O-aryl, C(O)O-cycloalkyl, C(O)alkyl, C(O)aryl, and C(O)cycloalkyl;
wherein R3 is a chemical moiety selected from hydrogen, alkyl, aryl, heteroaryl, cycloalkyl, cycloheteroalkyl, C(O)O-alkyl, C(O)O-aryl, C(O)O-cycloalkyl, C(O)alkyl, C(O)aryl, C(O)cycloalkyl, 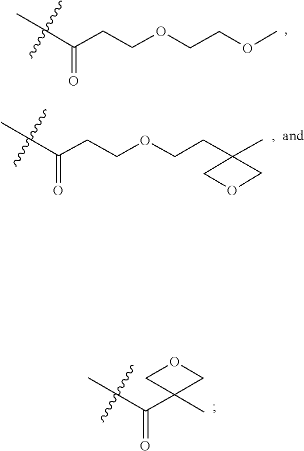
wherein R4 is a chemical moiety selected from hydrogen, alkyl, aryl, heteroaryl, cycloalkyl, cycloheteroalkyl, C(O)O-alkyl, C(O)O-aryl, C(O)O-cycloalkyl, C(O)alkyl, C(O)aryl, C(O)cycloalkyl, 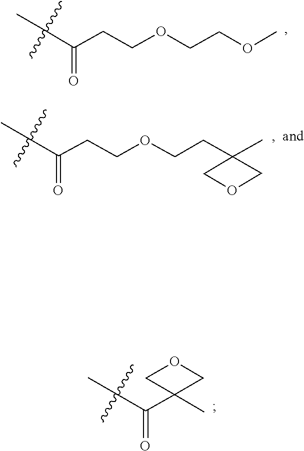
wherein R5 is a chemical moiety selected from alkyl, aryl, heteroaryl, cycloalkyl, cycloheteroaryl, wherein R5 can be substituted with one or more of the following: alkoxy, alkylamino, hydroxy, amino, alkyl, aryl, cycloalkyl, CO₂-alkyl, C(O)NR⁶R⁷, 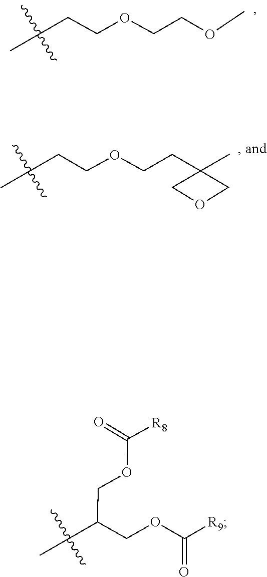
wherein R6 and R7 are each independently selected from the group consisting of hydrogen, alkyl, and cycloalkyl; or wherein R6 and R7 together form a C₄-C₅ alkyl or alkylene chain and together with the nitrogen to which they are attached form a 5 or 6 membered ring;
wherein R8 and R9 are independently selected from the group consisting of alkyl, alkenyl, cycloalkyl, cycloalkenyl, and aryl.

3-5. (canceled)

6. The composition of claim 2, wherein the prodrug compound is selected from:

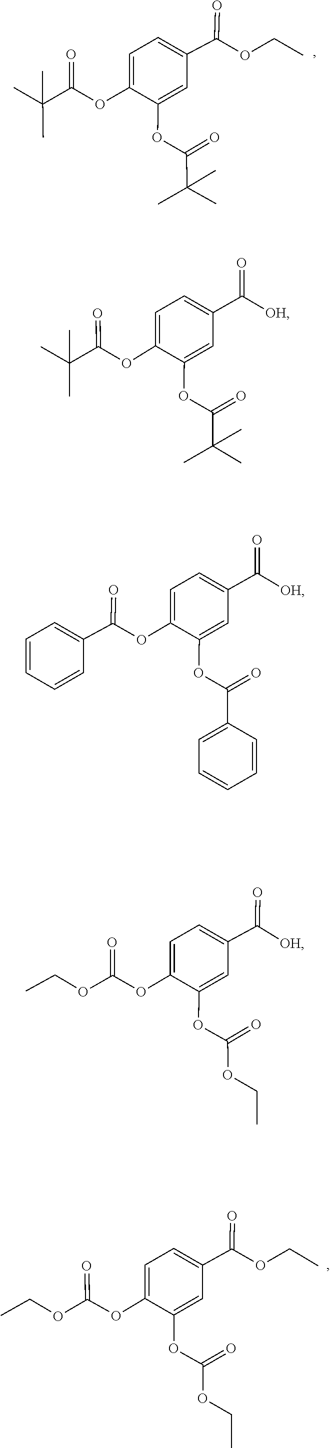

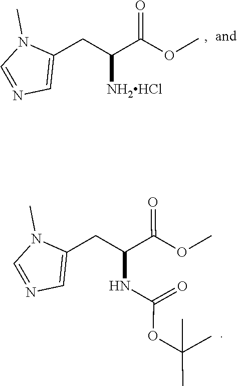

7. The composition of claim 1, wherein the metabolite is associated with (e.g., complexed, conjugated, encapsulated, absorbed, adsorbed, admixed).

8-10. (canceled)

11. The composition of claim 7, wherein the biodegradable agent is a microparticle or nanoparticle.

12. The composition of claim 11,
wherein the size of the microparticle is between 0.5 microns to 100 microns; and/or
wherein the average size of the nanoparticle is between 6 to 500 nm.

13. (canceled)

14. The composition of claim 11, wherein the nanoparticle is selected from the group consisting of sHDL nanoparticle, fullerenes, endohedral metallofullerenes buckyballs, trimetallic nitride templated endohedral metallofullerenes, single-walled and multi-walled carbon nanotubes, branched and dendritic carbon nanotubes, gold nanorods, silver nanorods, single-walled and multi-walled boron/nitrate nanotubes, carbon nanotube peapods, carbon nanohorns, carbon nanohorn peapods, liposomes, nanoshells, dendrimers, any nanostructures, microstructures, or their derivatives formed using layer-by-layer processes, self-assembly processes, or polyelectrolytes, microparticles, quantum dots, superparamagnetic nanoparticles, nanorods, cellulose nanoparticles, glass and polymer micro- and nano-spheres, biodegradable PLGA micro- and nano-spheres, gold nanoparticles, silver nanoparticles, carbon nanoparticles, iron nanoparticles, a modified micelle, metal-polyhistidine-DOPE@liposome, metal-polyhistidine-PEG, 4arm-PEG-polyhistidine-metal hydrogels, and sHDL-polyhistidine, and metal-organic framework (MOF) coordination polymer (CP).

15-16. (canceled)

17. The composition of claim 2,
wherein R1 is methyl;
wherein R2 is hydrogen or 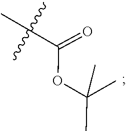 [SMILES: *C(=O)OC(C)(C)C]
wherein R3 is 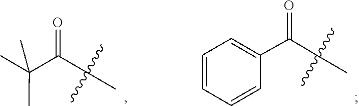
wherein R4 is 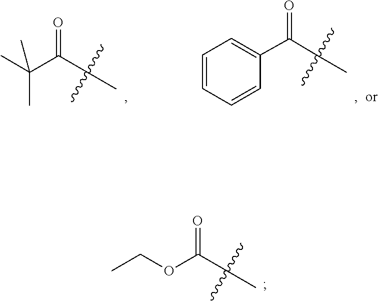
wherein R5 is hydrogen, or ethyl.

18-22. (canceled)

23. A method, comprising administration to a human subject 1) a cancer immunotherapy or vaccine, and 2) a composition as recited in claim 1;
wherein the method is for one or more of:
increasing the efficacy of a cancer immunotherapy or vaccine (e.g., cancer vaccine) (e.g., vaccines against infectious pathogens);
inhibiting the ability of a cancer cell to induce immune dysfunction;
treating or preventing cancer; and
increasing the efficacy of a vaccine;
wherein administration of the composition occurs prior to, concurrent with, and/or after administration of the vaccine (e.g., cancer vaccine) (e.g., vaccines against infectious pathogens) or cancer immunotherapy.

24-28. (canceled)

29. The method of claim 23, wherein the subject is a human suffering from or at risk of suffering from cancer.

30. The method of claim 23, wherein the cancer immunotherapy is one or more immune checkpoint inhibitor (ICI) inhibitors.

31. The method of claim 30, wherein the ICI inhibitors are selected from CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. ICIs include antibodies, or antigen binding fragments thereof, other binding proteins, biologic therapeutics or small molecules, that bind to and block or inhibit the activity of one or more of CTLA-4, PDL1, PDL2, PD1, BTLA, HVEM, TIM3, GAL9, LAG3, VISTA, KIR, 2B4, CD160 and CGEN-15049. Illustrative ICIs include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor). Checkpoint protein ligands include, but are not limited to PD-L1, PD-L2, B7-H3, B7-H4, CD28, CD86 and TIM-3. Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

32. The method of claim 23, wherein the vaccine is a vaccine for treating cancer, and/or a vaccine for treating and/or protecting from infectious pathogens.

33. The method of claim 23, wherein the cancer is any type of cancer responsive to cancer immunotherapy or cancer vaccine treatment.

34. The method of claim 23, wherein the cancer is one or more of breast, ovarian, prostate, lung, kidney, gastric, colon, testicular, head and neck, pancreas, brain, melanoma, and other tumors of tissue organs and hematological tumors, such as lymphomas and leukemias, including acute myelogenous leukemia, chronic myelogenous leukemia, chronic lymphocytic leukemia, T cell lymphocytic leukemia, and B cell lymphomas.

35. The method of claim 23, further comprising administering to the subject one or more chemotherapeutic agents selected from the group consisting of an alkylating agent, an antimetabolite, an anthracycline, an antitumor antibiotic, a monoclonal antibody, a platinum agent, a plant alkaloid, a topoisomerase inhibitor, a vinca alkaloid, a taxane, and an epipodophyllotoxin.

36. The method of claim 23, wherein the composition is administered orally (e.g., oral gavage), intratumorally, topically, intravenously, or subcutaneously.

37. A kit comprising a composition recited in claim 1, and one or more of a vaccine (e.g., cancer vaccine) (e.g., vaccines against infectious pathogens), and a cancer immunotherapy (e.g., an ICI inhibitor).
